(19) United States
(12) Patent Application Publication
(10) Pub. No.: US 2026/0130926 A1
(43) Pub. Date: May 14, 2026

(54) Advanced cannabinoid formulation for targeted management of Multiple Sclerosis

(71) Applicants: Dante Picazo, Houston, TX (US); Nancy Paola Duarte Delgado, Bogotà (CO); Nancy Rodrigues Das Fontes, Tondela (PT); Stephanie Dona Hartmann, Caracas (VE); Ana Maria Maldonado Vacca, Bogotà (CO); Maria Angelica Clavijo Romero, Santa Marta (CO); Rosangel del Valle Andrades Fuentes, Caracas (VE)

(72) Inventors: Dante Picazo, Houston, TX (US); Nancy Paola Duarte Delgado, Bogotà (CO); Nancy Rodrigues Das Fontes, Tondela (PT); Stephanie Dona Hartmann, Caracas (VE); Ana Maria Maldonado Vacca, Bogotà (CO); Maria Angelica Clavijo Romero, Santa Marta (CO); Rosangel del Valle Andrades Fuentes, Caracas (VE)

(21) Appl. No.: 18/942,512

(22) Filed: Nov. 9, 2024

(51) Int. Cl. A61K 31/00; A61K 9/00; A61K 9/107; A61K 31/593; A61K 36/07; A61K 36/185; A61K 47/26

(52) U.S. Cl. CPC A61K 31/658; A61K 9/006; A61K 9/1075; A61K 31/593; A61K 36/07; A61K 36/3482; A61K 47/26

(57) ABSTRACT

This disclosure presents an advanced oral cannabinoid formulation in drop form for fast-acting relief of multiple sclerosis (MS), utilizing absorption through the oral mucosa to enhance bioavailability. The formulation includes cannabinoids like CBD, CBG, and THC, combined with Hericium erinaceus extract and Vitamin D to boost therapeutic efficacy. It targets various MS subtypes, including relapsing-remitting (RRMS), secondary progressive (SPMS), primary progressive (PPMS), and progressive-relapsing (PRMS), addressing symptoms such as fatigue, motor issues, spasticity, cognitive impairments, and mood disorders. The cannabinoids activate CB1 and CB2 receptors, reducing excitatory signaling, muscle spasticity, and neuroinflammation. CBD and CBG further enhance neuroprotection by modulating dopamine receptor expression and uptake, promoting synaptic health. Hericium erinaceus and Vitamin D complement these effects, reducing neural and immune damage and improving mental well-being, providing a comprehensive therapeutic strategy for managing MS.

### FIELD OF THE DISCLOSURE

The technical field of the disclosure relates to the medicinal, therapeutic, and pharmaceutical uses of cannabinoids for managing multiple sclerosis (MS). This disclosure introduces therapeutic compositions featuring a broad-spectrum cannabinoid blend, including cannabidiol (CBD), cannabigerol (CBG), and tetrahydrocannabinol (THC), enhanced by a plant-based herbal extract and essential vitamins to support neurological health and reduce neuroinflammation.

Key ingredients include Hericium erinaceus (Lion's Mane) for its neuroprotective properties and Vitamin D to bolster immune function and promote remyelination. This composition targets the endocannabinoid system and inflammatory pathways while offering a natural, comprehensive strategy for managing MS symptoms. Through the synergistic effects of these bioactive compounds, the formulation aims to alleviate neuroinflammation, support cognitive function, and improve overall quality of life, providing a balanced, plant-based approach to MS management while minimizing the risks associated with conventional therapies.

### BACKGROUND INFORMATION

Multiple sclerosis (MS) is a chronic autoimmune neurological disease that targets myelinated axons within the central nervous system (CNS), leading to variable damage to both myelin and axons. It affects around 400,000 people in the U.S. and 2.5 million worldwide. It is more prevalent in women and typically appears between ages 20 and 40, though it can occur at any age. It is driven by gene-environment interactions, presenting a wide range of symptoms and disease progression patterns (Dighriri et al., 2023).

The clinical presentation of MS varies widely depending on lesion location within the CNS. The disease typically begins with a clinical attack known as Clinically Isolated Syndrome (CIS), manifesting in 85% of patients as optic neuritis, myelitis, or syndromes affecting the brainstem, cerebellum, or cerebral hemispheres. Relapsing-remitting MS (RRMS) involves recurrent relapses lasting at least 24 hours, while Primary Progressive MS (PPMS) and Secondary Progressive MS (SPMS) show gradual, progressive disability. Common symptoms include visual impairments, sensory issues (e.g., paresthesia and Lhermitte sign), motor symptoms (e.g., spasticity, paresis), brainstem and cerebellar dysfunction, and bladder, bowel, and sexual dysfunction. Cognitive impairment affects 40-70% of patients, often worsening in progressive forms, alongside fatigue, affective disturbances, and pain. The Expanded Disability Status Scale (EDSS) is commonly used to grade disability severity in MS, covering key functional domains such as pyramidal, cerebellar, and sensory functions (Filippi et al., 2018).

In all MS types, the primary pathological feature is the formation of focal plaques, or demyelinated lesions, around postcapillary venules. This process involves blood-brain barrier (BBB) breakdown, likely driven by pro-inflammatory cytokines and chemokines (e.g., TNF, IL-1B, IL-6) from resident and endothelial cells. The compromised BBB allows immune cells such as macrophages, T cells, and B cells to enter the central nervous system (CNS), leading to inflammation, further demyelination, oligodendrocyte loss, and neuro-axonal degeneration. These plaques affect both white and gray matter throughout the CNS, including the brain, optic nerve, and spinal cord. While white matter lesions correlate moderately with MS disability, other processes, like gray matter lesions and damage to normal-appearing brain tissue, contribute significantly to clinical impairment (Filippi et al., 2018).

MS treatment involves a variety of strategies aimed at managing symptoms, modifying disease progression, and improving quality of life. Disease-modifying therapies (DMTs) are crucial in the treatment, including interferon beta (such as Avonex and Rebif), glatiramer acetate (Copaxone), and newer agents such as ocrelizumab (Ocrevus) and siponimod (Mayzent). These therapies modulate the immune system to reduce inflammation and prevent new lesions from forming. Symptomatic treatments address specific problems, such as corticosteroids for relapses, antispasmodics for muscle spasticity, and medications for neuropathic pain, fatigue, and bladder dysfunction. Rehabilitative therapies, including physical and occupational therapy, are crucial in maintaining function and independence. These treatments, however, can lead to significant adverse effects.

Monoclonal antibodies may cause severe allergic reactions, including facial, lip, tongue, or throat swelling, which can obstruct breathing. Other potential side effects include hives, severe rash, mood changes such as depression or suicidal thoughts, and liver issues like elevated liver enzymes, hepatitis, or, in rare cases, liver failure. Autoimmune reactions, such as thyroiditis or Sjogren's syndrome, pancytopenia, cardiac issues like palpitations or arrhythmias, and neurological symptoms like muscle pain, weakness, or, in rare cases, seizures, have also been reported. Respiratory issues, including difficulty breathing or persistent cough, may occur, emphasizing the need for safer and more effective therapies to enhance patient outcomes while minimizing risks (Hauser et al., 2020).

Considering the limitations of current treatments for multiple sclerosis, particularly their side effects and the need for more effective options, a comprehensive approach utilizing CBD, CBG, and THC isolate, which are recognized for their anti-inflammatory and neuroprotective properties. Studies suggest that cannabinoids can modulate immune responses and reduce neuroinflammation, providing symptomatic relief for MS patients. These cannabinoids are enhanced by adding Hericium erinaceus, known for its neuroregenerative properties, and vitamin D, crucial in maintaining overall brain health.

This comprehensive strategy leverages the synergistic effects of various bioactive compounds, promoting an integrative approach to MS management while minimizing the risks associated with conventional pharmacological treatments.

### SUMMARY OF THE DISCLOSURE

The present disclosure relates to an advanced cannabinoid-based therapeutic composition, methods of producing the composition, and methods of treating multiple sclerosis (MS) using this composition. The primary embodiment of the invention is a pharmaceutical composition comprising a synergistic blend of cannabinoids, including cannabidiol (CBD), cannabigerol (CBG), and tetrahydrocannabinol (THC), combined with a neuroprotective herbal extract such as Hericium erinaceus and essential vitamins like Vitamin D. This formulation is designed to enhance neurological health, reduce neuroinflammation, and support the immune system, addressing key symptoms of MS.

The second embodiment pertains to the method of producing the composition, which involves creating a nanoemulsion through ultrasonic cavitation or high-pressure homogenization. This process ensures uniform dispersion of the active ingredients, enhances bioavailability, and stabilizes the formulation for effective oral administration.

The third embodiment covers the method of treatment for multiple sclerosis, utilizing the disclosed composition for the targeted management of symptoms such as spasticity, cognitive impairments, chronic pain, and fatigue. The treatment involves administering the composition as oral drops, leveraging the enhanced bioavailability of the nanoemulsion to deliver consistent and effective relief. The formulation aims to modulate the endocannabinoid system and inflammatory pathways, providing a natural, integrative approach to improving patient outcomes and quality of life in individuals with MS.

Together, these embodiments present a comprehensive solution for managing multiple sclerosis, offering a safer and more effective alternative to conventional therapies by utilizing a combination of cannabinoids, neuroprotective extracts, and essential vitamins. The disclosed invention provides a versatile platform adaptable to various dosage forms and administration routes, tailored to meet the diverse needs of patients with MS.

### DETAILED DESCRIPTION

### Definitions

Multiple sclerosis (MS) is a complex autoimmune neurological disease that is characterized by demyelinating lesions accumulating in both the white and gray matter of the brain and spinal cord (Filippi et al., 2018). MS presents in various forms, each with distinct clinical features and progression patterns. The initial presentation may be classified as Clinically Isolated Syndrome (CIS), which involves neurological symptoms lasting at least 24 hours and has the potential to progress to MS. The primary subtypes of MS include the following:

Clinically Isolated Syndrome (CIS): The initial presentation with neurological symptoms lasting at least 24 hours, potentially leading to MS.

Relapsing-Remitting MS (RRMS): The most common form, marked by episodes of neurological symptoms (relapses) that fully or partially resolve (remission).

Secondary Progressive MS (SPMS): Follows RRMS, with progressive worsening of neurological function over time and accumulating disability.

Primary Progressive MS (PPMS): A rarer form, presenting as gradual progression of disability from onset without initial relapses.

Each subtype may be classified as active or non-active based on relapses or MRI-detected lesion activity.

The disease usually presents between 20-40 years of age, affecting more frequently women (60%) than men (40%).

Pathophysiologically, the process begins with autoreactive T cells mistakenly identifying myelin as an antigen, initiating an inflammatory cascade within the central nervous system (CNS). The release of interferon-gamma, IL-17, and granulocyte-macrophage colony-stimulating factors by these T cells induces the production of proinflammatory cytokines such as TNF-α, IL-1, IL-2, IL-12 and IL-17, as well as chemokines. These inflammatory molecules attract and activate macrophages and microglia, perpetuating the inflammatory process.

Simultaneously, type 2 helper T cells (Th2) release anti-inflammatory cytokines to help regulate the immune response and stimulate B cell proliferation and antibody production. These antibodies, together with other immune system components, such as complement, manage to cross the damaged blood-brain barrier and participate in the inflammatory response within the central nervous system. Chronic inflammation damages myelin and axons, forming the characteristic demyelinating plaques associated with multiple sclerosis. The loss of myelin disrupts nerve conduction and, in the long term, can lead to neuronal degeneration (Dighriri et al., 2022).

During the disease, most of the neurological functional systems are usually affected (pyramidal, sensory, cerebellar, brain stem, sphincter, visual, mental), with motor (90%), sensory (77%) and cerebral (75%) alterations being the most frequent, followed in decreasing order by the brain stem, sphincter, mental and visual alterations (Fernandez et al., 2015).

Cannabinoids (endocannabinoids, phytocannabinoids, or synthetic cannabinoids) can regulate these processes at different levels. Neuroprotection has generally been associated with the action of cannabinoids by activating the CB1 receptor and modulating the inflammatory response to the activation of the CB2 receptor. Therefore, they are a good as alternative immunomodulatory/neuroprotective therapeutic agents in diseases such as MS.

The Endocannabinoid System (ECS) comprises cannabinoid receptors, endocannabinoids, and enzymes that regulate the production and release of endocannabinoids, causing cell reactions. When an alteration occurs, the human body synthesizes endocannabinoids that usually interact with cannabinoid receptors. This interaction activates molecular pathways within cells that cause stability in vital and physiological processes (Di Marzo & De Petrocellis, 2006).

Likewise, it can modulate muscle tone and reduce spasticity by acting on presynaptic CB1 receptors to reduce glutamatergic transmission (Baker & Pryce, 2008).

To date, the cannabinoid receptors CB1 and CB2 are the best characterized. They differ in how they transmit the signal and their distribution in different tissues. Both are G protein-coupled receptors (GPCRs) and their activation leads to the inhibition of adenylyl cyclase, which prevents the conversion of ATP to cyclic AMP (CAMP) and prevents specific voltage-sensitive calcium channels, in addition to stimulating mitogen-activated protein kinases (MAP kinases) and inwardly rectifying potassium channels (GIRKs), and recruiting beta-arrestins, among other actions (Howlett & Abood, 2017).

At the molecular level, cannabinoids exert therapeutic effects by activating CB1 and CB2 receptors.

CB1 predominates in the central nervous system (CNS), specifically in astrocytes and astroglia, and is also expressed at lower levels in many peripheral tissues. Activation of these receptors may inhibit the release of proinflammatory cytokines and reduce immune cell infiltration into the CNS.

The CB2 receptor is expressed at high levels in leukocytes. Still, there is also evidence of limited CB2 receptor expression in activated microglial cells, and the level of expression depends on the degree of activation of these cells. These help regulate the immune response and reduce the chronic inflammation characteristic of multiple sclerosis (Mestre et al., 2006).

In the research of Baker et al., 2000, cannabinoids were shown to improve muscle spasticity through mechanisms mediated by both CB1 and CB2 receptors.

Cannabinoid (CB) receptor agonism using full agonists has been shown to quantitatively improve both tremor and spasticity in diseased mice. The worsening of these symptoms following antagonism of CB1 and CB2 receptors, particularly the CB1 receptor, suggests that the endogenous cannabinoid system may be tonically active in controlling tremor and spasticity, as these receptors act presynaptically to reduce glutamate release. This supports the rationale for the therapeutic potential of cannabis in managing MS symptoms.

Tetrahydrocannabinol (Δ⁹-THC) is a partial CB1 agonist but provides more limited CB2 agonist activity; these results suggest that the effect on tremor is mediated primarily by the brain CB1 receptor since activation of the cannabinoid receptor type 1 (CB1) reduces glutamatergic transmission in animals exposed to Δ⁹-tetrahydrocannabinol (THC), and chronic cannabis use reduces glutamate metabolites in human brains (Baker & Pryce., 2008).

Cannabinoid treatments show promising results in managing multiple sclerosis (MS) symptoms, particularly spasticity and neuropathic pain. Oral cannabis extracts, nabiximols, and synthetic Δ⁹-THC demonstrate efficacy in reducing patient-reported spasticity and alleviating MS-related pain, although they do not consistently improve physician-rated measures of spasticity. Cannabinoids are well-tolerated, making them a promising therapeutic option for managing MS symptoms with minimal risk of adverse effects (Rice & Cameron, 2018).

Cannabinoids exhibit neuroprotective effects in multiple sclerosis (MS) by modulating excessive neuronal excitability within the central nervous system (CNS). The CB1 receptors, primarily located on GABAergic and glutamatergic neurons, regulate neurotransmitter release, helping balance excitatory and inhibitory transmission and alleviating symptoms like spasticity, pain, and tremors. Cannabinoids stimulate endocannabinoid release and activate CB1 receptors, reducing neurotransmitter release at presynaptic terminals and thereby decreasing neuronal hyperactivity. They also influence microglial function through CB2 receptors, which are upregulated during inflammation and help mitigate pro-inflammatory responses by reducing TNF-α and oxidative radicals.

Cannabidiol (CBD) provides additional neuroprotection due to its antioxidant properties, which combat oxidative stress, a key factor in neurodegeneration. Studies show CBD's potency in scavenging reactive oxygen species (ROS), reducing lipid peroxidation, and lowering cell apoptosis markers like DNA fragmentation, underscoring its potential as a neuroprotective agent in MS (Nouh et al., 2024).

Cannabinoids have shown potential for modulating pain pathways in patients with Secondary Progressive Multiple Sclerosis (SPMS), impacting both central and peripheral nervous systems. Treatment resulted in reduced pain sensitivity and increased pain thresholds, likely due to cannabinoids' ability to influence nociceptive signal transmission. Cannabinoid receptors appear to play a key role in pain modulation by regulating neurotransmitter release and decreasing inflammatory signaling, suggesting that cannabinoids may help manage chronic pain in MS patients by addressing the physiological mechanisms underlying nociception (Conte et al., 2009).

Cannabidiol (CBD) has garnered significant attention for its potential therapeutic effects in neurological disorders, including epilepsy, multiple sclerosis, and neurodegenerative diseases. It exerts its action through various molecular targets, including ion channels, receptors, enzymes, and proteins involved in neuroinflammation and cell signaling. CBD interacts with the endocannabinoid and non-endocannabinoid systems, affecting pathways regulating calcium, neurotransmitter release, and oxidative stress. CBD's effects are often mediated through mechanisms beyond its interaction with CB1 and CB2 receptors, such as modulation of transient receptor potential (TRP) channels and peroxisome proliferator-activated receptors (PPARs). Despite these challenges, CBD's ability to modulate complex neurophysiological processes suggests it may hold promise as a treatment for various neurological conditions (Ibeas Bih et al., 2015).

Cannabidiol (CBD) interacts with cannabinoid receptors (CB1 and CB2) and non-cannabinoid receptors like TRPV1 and exhibits immunomodulatory and pain-relieving properties. It modulates immune responses by inhibiting pro-inflammatory cytokines and limiting immune cell activation, potentially reducing inflammation and exacerbating conditions like multiple sclerosis (MS). In the context of pain, CBD activates TRPV1 receptors on dorsal root ganglion neurons, leading to their desensitization and a reduction in pain sensitivity. This is achieved by modulating calcium ion flow and altering downstream pathways involved in pain perception and neuronal excitability. By decreasing the hyperactivity of these neurons, CBD may alleviate chronic pain, particularly inflammatory and neuropathic pain. While these mechanisms offer promise, further clinical trials are needed to confirm CBD's efficacy and establish optimal dosing for therapeutic use in MS and chronic pain management (Anand et al., 2020; Furgiuele et al., 2021).

Cannabinoids exert an anti-inflammatory and neuroprotective action by inducing the production of IL-1ra, a molecule that limits the action of IL-1B, a key cytokine in inflammation. The cannabinoid system also blocks the release of cytotoxic agents and proinflammatory cytokines. On the other hand, it promotes migration and regulates cell proliferation, limiting the inflammatory process and preventing cell spread and damage. These combined effects suggest that cannabinoids could be useful in reducing inflammation, protecting neurons, and improving symptoms associated with multiple sclerosis (Mestre et al., 2006).

Additionally, CBD, along with CBG, both at the proposed doses, can increase the expression of dopaminergic receptors 2 and 4 in NSC-34 cells while simultaneously inhibiting the expression of monoamine transporter 2, thereby reducing dopamine uptake. This may also play a role in synaptic plasticity.

Cannabigerol (CBG) exerts its function by acting with the CB2 receptors, where its effect has been associated with the modulation of the levels of the cytokines IL-1β, IL-10, and IFNγ, the decrease in the expression of INOS, and the production of nitric oxide, exerting a neuroprotective, anti-inflammatory, and antioxidant effect (Carrillo-Salinas, 2016).

Additionally, a novel quinone derivative of cannabigerol (CBG) demonstrates potential for reducing neuroinflammation in a chronic multiple sclerosis (MS) model. This compound shows anti-inflammatory effects by modulating immune responses, notably decreasing the activity of proinflammatory markers such as TNF-α, IL-1β, and IL-6. Additionally, it promotes the survival of oligodendrocytes, which are crucial for nerve function and repair. By interacting with both cannabinoid receptors and peroxisome proliferator-activated receptor-gamma (PPAR-γ), this CBG quinone not only dampens inflammatory responses but also supports neuroprotection, suggesting its promise as a therapeutic candidate for managing chronic neuroinflammatory conditions like MS (Granja et al., 2012).

Hericium erinaceus, also known as lion's mane mushroom, belongs to the Basidiomycota phylum within the Hericiaceae family. This neuroprotective action refers to the ability of substances or factors to protect and support the health of nerve cells and prevent their damage. Research shows that H. erinaceus exhibits numerous therapeutic properties, such as antioxidant, anti-inflammatory, and neuroprotective, as it induces the synthesis of nerve growth factor (NGF) through its compounds (hericenones and erinacines), antioxidant and anti-inflammatory because it inhibits the cytotoxicity of β-amyloid (Aβ) and protects nerve cells from death caused by oxidative stress or endoplasmic reticulum stress (Szucko-Kociuba et al., 2023).

Hericium erinaceus mycelium and its small bioactive compounds have been found to support the maturation of oligodendrocytes, cells responsible for forming myelin sheaths in the central nervous system. This process is associated with increased production of myelin essential protein (MBP), a key component of myelin. By promoting oligodendrocyte development and myelination, Hericium erinaceus could hold potential therapeutic benefits for neurological conditions characterized by demyelination, offering a natural means to enhance brain health and repair mechanisms (Huang et al., 2021).

Vitamin D is mainly acquired through skin exposure to sunlight and dietary intake. A potential role of Vitamin D in myelination and remyelination has been suggested since Vitamin D decreases the expression of inducible nitric acid synthase in microglia; it could influence the inflammatory-anti-inflammatory balance relevant to remyelination. Vitamin D also increases microglial activation, which could facilitate the removal of myelin debris and remyelination. (Matías-Guíu et al., 2016).

### Pharmaceutical Composition

Aspects of the disclosure provide a methodology to address the main symptoms associated with multiple sclerosis to obtain the optimal balance of health in patients using a nano platform composition of phytocannabinoids (CBD, CBG, and THC isolates) and natural extracts such as Hericium erinaceus (lion's mane) and Vitamin D, specifically targeting patients who have a diagnosis of multiple sclerosis.

The present disclosure discloses a composition designed for the treatment of multiple sclerosis, specifically formulated to include a combination of active and stabilizing ingredients aimed at addressing the complex symptomatology of the disease. The composition features a primary active principle consisting of at least one cannabinoid, selected for its potent anti-inflammatory, neuroprotective, and analgesic properties, which are particularly effective in managing the chronic pain, spasticity, and neuroinflammation associated with multiple sclerosis. The second active principle is an herbal extract, chosen for its neuroregenerative and antioxidant capabilities, contributing to the protection and repair of neural tissues, while also modulating the immune response. The third active principle comprises a vitamin, which plays a crucial role in supporting overall neurological health and enhancing the body's ability to maintain myelin integrity and immune balance, both of which are essential for patients with multiple sclerosis.

To ensure consistent delivery and stability of the formulation, the composition includes two emulsifying agents that facilitate the formation of a stable emulsion, particularly in an oral dosage form. These emulsifiers work synergistically to maintain the homogeneity of the mixture, preventing phase separation and ensuring that the active principles remain evenly dispersed. The inclusion of emulsifying agents also enhances the solubility and bioavailability of the hydrophobic components, such as cannabinoids, improving their absorption when administered orally. This well-balanced formulation is tailored for oral administration, offering a convenient and effective therapeutic option that targets the key symptoms and underlying pathophysiological mechanisms of multiple sclerosis, providing sustained symptom relief and supporting neurological health.

The composition further specifies that the cannabinoids incorporated as the primary active principle are selected from cannabidiol (CBD), tetrahydrocannabinol (THC), cannabigerol (CBG), or combinations thereof. Each of these cannabinoids is chosen for its distinct pharmacological profile and contribution to the overall therapeutic efficacy of the formulation. CBD is well-regarded for its strong anti-inflammatory and neuroprotective properties, making it effective in reducing neuroinflammation and oxidative stress, which are key factors in the progression of multiple sclerosis. THC, included at controlled concentrations, provides additional benefits in managing symptoms such as spasticity, chronic pain, and muscle rigidity, while minimizing psychoactive effects. CBG complements the therapeutic action by offering potent neuroprotective and immune-modulating effects, aiding in the repair of damaged neural tissues and enhancing overall neurological function.

When used in combination, these cannabinoids exhibit synergistic effects, amplifying their individual benefits through the entourage effect. This synergistic action results in improved symptom relief and a broader therapeutic impact, targeting multiple pathways involved in the pathophysiology of multiple sclerosis. By selecting CBD, THC, and CBG, or a tailored combination of these cannabinoids, the composition offers a comprehensive approach to managing the diverse and complex symptoms of multiple sclerosis, enhancing patient outcomes and providing an effective, versatile treatment option.

In an embodiment, the composition specifies that the cannabinoids—CBD, THC, CBG, or combinations thereof—are included at concentrations ranging from 0.2% to 30.0%. This concentration range is designed to provide flexibility in formulating the composition, allowing it to be tailored according to the therapeutic needs and symptom severity of individual patients with multiple sclerosis. The lower end of the range (0.2%) offers a milder dosage suitable for patients who may be sensitive to cannabinoids or those requiring minimal psychoactive effects, particularly in cases where symptom control can be achieved with lower doses.

On the other hand, higher concentrations (up to 30.0%) are intended for more severe cases or for patients who require stronger anti-inflammatory and neuroprotective effects to manage advanced symptoms of multiple sclerosis, such as severe spasticity, chronic pain, and neurodegeneration. This range also allows for the formulation of varying cannabinoid ratios, optimizing the therapeutic potential through combinations of CBD, THC, and CBG. The ability to adjust the concentration within this range provides a customizable treatment option, ensuring that the dosage can be tailored to meet individual patient needs, maximize efficacy, and minimize potential side effects, while enhancing the overall therapeutic experience.

In an embodiment, the composition further defines the concentration ranges for the herbal extract and the vitamin, specifying that the herbal extract is present at a concentration between 20.0% and 30.0%, while the vitamin is included in the range of 0.01% to 0.02%. The specified concentration range for the herbal extract ensures that it is present at a therapeutically effective dose, contributing significantly to the formulation's neuroprotective and anti-inflammatory benefits. This range, particularly when selecting potent extracts such as Hericium erinaceus (Lion's Mane), allows for sufficient levels of bioactive compounds like erinacines and hericenones, which are known to support nerve growth, reduce oxidative stress, and promote cognitive function, all of which are crucial in managing multiple sclerosis symptoms.

The inclusion of the vitamin, typically Vitamin D, at concentrations between 0.01% and 0.02%, is strategically chosen to complement the effects of the cannabinoids and herbal extract. This vitamin plays a critical role in immune modulation, supporting myelin repair, and enhancing overall neurological health. Even at low concentrations, the vitamin contributes to the regulation of immune responses and helps mitigate neuroinflammatory processes associated with multiple sclerosis. By defining these specific concentration ranges, the composition ensures that the active ingredients are present at optimal levels, maximizing their therapeutic potential while maintaining a balanced formulation that is well tolerated by patients. This precise dosage framework allows the treatment to be effective across a broad spectrum of symptom severities, offering a comprehensive and integrative approach to the management of multiple sclerosis.

In an embodiment, the composition specifies the use of emulsifying agents selected from Polysorbate 80, Sorbitan 20, or combinations thereof, incorporated at concentrations ranging from 2.0% to 10.0%. These emulsifying agents are integral to the formulation, as they facilitate the creation of a stable oil-in-water emulsion, which is crucial for the effective delivery of lipophilic active ingredients like cannabinoids. The inclusion of Polysorbate 80 and Sorbitan 20 ensures that the hydrophobic cannabinoids and other lipid-based components are evenly dispersed within the aqueous phase, preventing phase separation and maintaining the homogeneity of the mixture.

Polysorbate 80 is particularly effective as a solubilizing agent, enhancing the bioavailability of the cannabinoids by reducing the surface tension between the oil and water phases. This property aids in the consistent absorption of the active ingredients, ensuring reliable therapeutic effects with each dose. Sorbitan 20 acts synergistically with Polysorbate 80, contributing to the stabilization of the emulsion and providing additional emulsifying power that supports the uniform distribution of all components.

The specified concentration range of 2.0% to 10.0% is designed to provide flexibility in formulation, allowing adjustments based on the desired consistency, stability, and viscosity of the final product. Lower concentrations are suitable for formulations requiring a thinner consistency, while higher concentrations enhance the stability of the emulsion, particularly in formulations intended for prolonged storage or exposure to varying temperatures. This careful selection and concentration range of emulsifying agents ensure that the composition remains stable, effective, and well-suited for oral administration, optimizing the delivery and bioavailability of the active ingredients in the treatment of multiple sclerosis.

In an embodiment, the composition is further characterized as a nanoplatform, featuring an advanced nanoemulsion system with an average particle size of less than 200 nm. This nanoscale particle size is a critical aspect of the formulation, as it significantly enhances the bioavailability of the active ingredients, particularly the hydrophobic cannabinoids, by increasing their surface area and solubility. The reduced particle size allows the cannabinoids, herbal extracts, and vitamins to be absorbed more efficiently across biological membranes, facilitating faster onset of therapeutic effects.

The nanoplatform is specifically designed to enable the controlled release of the active ingredients, providing a sustained and gradual delivery over an extended period. This controlled release profile is advantageous in managing chronic conditions such as multiple sclerosis, where consistent therapeutic levels of cannabinoids are essential for effective symptom relief. By ensuring a steady release, the formulation minimizes fluctuations in the plasma concentration of the active ingredients, reducing the need for frequent dosing and enhancing patient adherence.

The nanoemulsion system also contributes to the stability of the composition, preventing the aggregation of nanoparticles and maintaining a uniform dispersion of the active components throughout the formulation. This stability, combined with the controlled release mechanism, optimizes the therapeutic efficacy of the composition, providing prolonged relief from symptoms and targeting the underlying pathophysiological processes of multiple sclerosis. The use of a nanoplatform with an average particle size of less than 200 nm represents a significant advancement in drug delivery technology, enhancing both the performance and reliability of the treatment.

In an embodiment, the composition is provided as an oral nanoemulsion dosage, specifically formulated in the form of drops. This dosage form leverages the benefits of nanoemulsion technology, which enhances the solubility and bioavailability of the active ingredients, particularly the hydrophobic cannabinoids such as CBD, CBG, and THC. The nanoemulsion is designed to be administered sublingually or orally, allowing for efficient absorption through the mucosal membranes, bypassing first-pass metabolism and leading to faster onset of therapeutic effects.

The drop formulation offers precise dosing, allowing healthcare providers and patients to easily adjust the dosage based on individual therapeutic needs. The uniform particle size of the nanoemulsion ensures that each drop delivers a consistent concentration of the active ingredients, including cannabinoids, herbal extracts, and vitamins, optimizing their therapeutic potential. The small droplet size, typically less than 200 nm, facilitates rapid absorption and efficient transport across biological barriers, improving the delivery of the active compounds to the central nervous system.

The oral drop format is particularly advantageous for patients with multiple sclerosis, as it provides a user-friendly and convenient method of administration that can be easily incorporated into daily routines. This delivery form enhances patient compliance, offers flexible dosing, and ensures that the active ingredients are effectively delivered to the target sites, providing consistent symptom relief and supporting long-term management of the disease. The use of a nanoemulsion dosage in drop form represents a sophisticated approach to cannabinoid therapy, combining the benefits of advanced drug delivery technology with a practical and patient-centric administration method.

In an embodiment, the composition further specifies that the herbal extract is Hericium erinaceus (commonly known as Lion's Mane) and the vitamin component is Vitamin D. The inclusion of Hericium erinaceus, a well-known neuroprotective extract, is particularly advantageous in the context of treating multiple sclerosis due to its ability to promote nerve growth factor (NGF) synthesis. The bioactive compounds in Hericium erinaceus, such as erinacines and hericenones, support neural regeneration, enhance cognitive function, and reduce oxidative stress, all of which are critical factors in managing the neurodegenerative aspects of multiple sclerosis.

Vitamin D is selected for its essential role in immune modulation and neurological health. Its presence in the composition aids in regulating immune responses, reducing inflammation, and promoting the repair of myelin, which is often damaged in patients with multiple sclerosis. Adequate levels of Vitamin D have been associated with improved outcomes in MS patients, as this vitamin helps mitigate the autoimmune processes that contribute to disease progression.

Together, Hericium erinaceus and Vitamin D complement the action of cannabinoids by addressing different aspects of multiple sclerosis pathology. While the cannabinoids primarily target pain relief, spasticity, and neuroinflammation, Hericium erinaceus enhances neuroprotective and regenerative effects, and Vitamin D supports immune balance and myelin integrity. This synergistic combination provides a comprehensive therapeutic approach, targeting both the symptomatic and underlying biological mechanisms of multiple sclerosis, enhancing patient outcomes, and supporting overall neurological health.

The details outlined in the examples above are broader than those shown. Modifications can be made without departing from the scope of the disclosure and the equivalent of the claims. The following description of specific embodiments will help clarify the construction, operation, and additional benefits of the disclosure.

The present disclosure relates to a composition designed for the treatment of multiple sclerosis, including a) an active ingredient comprising a cannabinoid, b) a second active ingredient comprising one herbal extract, c) an essential vitamin, and d) At least two emulsifying agents suitable for oral drops administration. In some embodiments, the active ingredient has at least one cannabinoid selected from several cannabinoids, provided in the form of CBD, CBG, and THC.

These cannabinoids can be used individually or in specific combinations to achieve specific therapeutic effects tailored to the patient's needs, such as the selection of cannabinoids such as CBD, CBG, THC or combinations thereof.

Functional magnetic resonance imaging (fMRI) has shown how CBD influences neural activity in regions critical for emotional and cognitive processes, such as the medial temporal cortex and striatum. These regions are often compromised in multiple sclerosis (MS) due to neuroinflammation and demyelination. CBD's modulation of these regions and its action on limbic and paralimbic areas involved in mood regulation may help alleviate MS-related cognitive and emotional issues.

The other compounds in the formula-CBG, THC, Hericium erinaceus, and Vitamin D-contribute synergistically to the overall neuroprotective and anti-inflammatory effects, targeting multiple pathways implicated in the pathology of multiple sclerosis (MS). Cannabigerol (CBG) acts primarily through the CB2 receptors, modulating immune responses and reducing the release of pro-inflammatory cytokines like TNF-α and IL-6, which are key drivers of neuroinflammation in MS. This modulation helps decrease oxidative stress, supporting neural integrity and potentially enhancing remyelination processes. Tetrahydrocannabinol (THC), as a partial agonist of CB1 receptors, further contributes by reducing excitatory neurotransmission, alleviating symptoms such as spasticity and tremors.

THC's interaction with CB1 receptors also aids in diminishing neuroinflammation and oxidative damage, two critical factors in the progression of MS. Hericium erinaceus, known for its neuroregenerative properties, enhances the formulation by promoting nerve growth factor (NGF) synthesis, which is essential for the repair and maintenance of neural tissues. Its compounds, such as hericenones and erinacines, have demonstrated the ability to protect neurons from oxidative stress and support oligodendrocyte function, which is vital for myelin sheath preservation. Vitamin D, integral for immune regulation and neurological health, plays a key role in modulating the inflammatory response and promoting myelination. Its deficiency has been associated with increased MS severity, and supplementation can help restore immune balance, reduce lesion formation, and support overall neural health.

Collectively, these ingredients offer comprehensive support for mental and neurological well-being in MS patients, as evidenced by fMRI studies demonstrating the positive effects of neuroprotective and anti-inflammatory therapies. Cannabidiol (CBD), as a core component of the formulation, has shown considerable promise in mitigating a range of MS-related symptoms, including cognitive deficits, muscle spasticity, chronic pain, and autonomic dysfunctions like bladder and bowel issues. By modulating the endocannabinoid system, CBD helps reduce the muscle rigidity often experienced as stiffness and spasticity, alleviates tremors, and addresses persistent fatigue, even in cases where rest does not provide relief. Its anxiolytic and mood-stabilizing effects contribute to improved attention and reduced irritability and frustration, while its influence on serotoninergic pathways has been linked to enhanced sleep quality.

CBD has been shown to support healthier sleep patterns, effectively managing symptoms like insomnia, depression, and disrupted sleep cycles that frequently accompany MS. Together, these bioactive compounds form a robust therapeutic approach, offering targeted relief from the complex, multifactorial symptoms of multiple sclerosis.

Isolated forms of cannabidiol (CBD), cannabigerol (CBG), and tetrahydrocannabinol (THC) are obtained by extracting these cannabinoids from the cannabis plant and removing all other compounds. Combined, these cannabinoids can produce an “entourage effect”—a synergistic interaction that enhances and maximizes each cannabinoid's therapeutic properties. This interaction may amplify their effectiveness in reducing nerve inflammation and oxidative stress, significantly relieving spasticity and other symptoms of multiple sclerosis.

In an additional embodiment, Hericium erinaceus, commonly referred to as Lion's Mane, serves as a potent neuroprotective agent within the composition. Its bioactive compounds, including hericenones and erinacines, are capable of stimulating the synthesis of nerve growth factor (NGF), which plays a critical role in the maintenance, survival, and regeneration of neurons. By enhancing NGF levels, Hericium erinaceus supports the growth of new neural connections and aids in the repair of damaged nerve cells, making it particularly beneficial for neurological disorders such as multiple sclerosis. Furthermore, this herbal extract helps regulate inflammatory pathways by inhibiting the production of pro-inflammatory cytokines, thereby reducing neuroinflammation, a hallmark of MS progression.

The antioxidative properties of Hericium erinaceus mitigate oxidative stress, one of the key factors contributing to neuronal degeneration, and protect nerve cells from apoptosis, thereby preserving neural integrity. Additionally, Hericium erinaceus has been shown to elevate the levels of NGF and catecholamines in critical brain regions such as the locus coeruleus and hippocampus, areas associated with cognitive function and mood regulation. This enhancement in neurochemical support contributes to increased neuronal survival, improved synaptic plasticity, and potentially better cognitive outcomes in patients with multiple sclerosis. By integrating Hericium erinaceus into the formulation, the composition leverages its multifaceted neuroprotective effects, aiming to support nerve health and enhance the brain's natural repair mechanisms.

The nanoplatform delivery system employed in this formulation optimizes the efficient transport of cannabinoids, herbal extracts, and vitamins, significantly enhancing their bioavailability and ensuring a controlled release profile. This advanced delivery method leverages nanoemulsion technology, which reduces particle size to the nanometer scale, increasing the surface area and improving the solubility of the hydrophobic active ingredients. As a result, the cannabinoids and herbal components can be absorbed more rapidly and uniformly across biological membranes, particularly through the oral mucosa, leading to faster onset of therapeutic effects.

The controlled release properties of the nanoplatform further allow for the gradual release of active ingredients, maintaining consistent therapeutic levels over an extended period. This sustained delivery is particularly advantageous in managing the chronic and fluctuating symptoms of multiple sclerosis, such as spasticity, neuropathic pain, cognitive impairments, and fatigue. By tailoring the composition of cannabinoids to target specific receptors and pathways involved in neuroinflammation and neuroprotection, the nanoplatform enhances the overall therapeutic efficacy. The precise delivery and controlled release reduce the frequency of dosing and improve patient adherence, offering a comprehensive and versatile approach to mitigating the symptoms and slowing the progression of multiple sclerosis and related neurodegenerative conditions.

In one embodiment of the disclosure, the cannabinoids include CBD, THC, CBG, or combinations thereof, and these cannabinoids are incorporated at concentrations ranging from 0.2% to 30.0%. This concentration range is designed to provide flexibility in formulating the composition, allowing adjustments based on therapeutic requirements and patient-specific factors. In various embodiments, the cannabinoid concentrations can be fine-tuned to enhance the therapeutic potency and optimize the patient's response.

For example, a formulation may include 10% CBD isolate, 5.0% CBG isolate, and 0.25% THC isolate, demonstrating a balanced and targeted approach to cannabinoid delivery. These specific ratios are chosen to offer a flexible framework that can be modified to increase or decrease the dosage as needed, accommodating individual patient needs, tolerance levels, and the severity of the condition being treated. This adaptability ensures precise dosage modifications, which can be tailored based on ongoing assessments of patient response and therapeutic goals, ultimately supporting a personalized treatment strategy that maximizes efficacy while minimizing potential side effects.

The ratio of cannabinoids within the composition is flexible and can be tailored to optimize the therapeutic profile while maintaining overall efficacy. This customizable approach allows for the development of specific formulation strategies aimed at targeting particular symptoms or addressing distinct physiological pathways associated with multiple sclerosis. For instance, increasing the proportion of CBD may enhance its well-documented anti-inflammatory and neuroprotective effects, making it particularly beneficial for reducing chronic pain and neuroinflammation.

Alternatively, adjusting the ratio to include higher concentrations of CBG can amplify its role in modulating immune responses and reducing oxidative stress, while increasing THC content may be advantageous for managing severe spasticity and enhancing muscle relaxation. In other scenarios, a balanced composition of CBD, CBG, and THC isolates can be designed to simultaneously address multiple symptoms, leveraging the unique and complementary mechanisms of each cannabinoid. The combined use of these cannabinoids offers a multifaceted therapeutic potential due to their synergistic effects on the endocannabinoid system, which plays a crucial role in maintaining neural homeostasis.

This balanced mixture is particularly effective for treating psychiatric and neurodegenerative conditions, including multiple sclerosis, by promoting positive neural changes and modulating neurotransmitter systems. The interaction of cannabinoids with CB1 and CB2 receptors helps to alleviate spasticity, reduce neuroinflammation, and mitigate symptoms of anxiety and depression often associated with relapses and disease progression. Furthermore, the formulation's impact on oxidative stress reduction contributes to the preservation of neuronal integrity and enhances overall patient well-being, offering a comprehensive therapeutic strategy that addresses both the physical and psychological aspects of multiple sclerosis.

By precisely modulating the concentrations and ratios of cannabinoids, the composition can be customized to offer alternative treatment options for multiple sclerosis, enhancing patient outcomes and overall well-being while avoiding the adverse effects typically seen with conventional pharmacological therapies. This flexibility allows the formulation to be adapted to the specific needs of individual patients, addressing the diverse symptomatology and progression patterns of MS. The incorporation of nanoplatform technology further enhances the efficacy of the composition by enabling controlled release of the active ingredients. This advanced delivery system ensures consistent therapeutic levels of cannabinoids, herbal extracts, and vitamins over an extended period, thereby reducing the need for frequent dosing and improving patient adherence.

The sustained release profile is particularly advantageous in the context of chronic conditions like multiple sclerosis, where prolonged symptom relief and stable blood levels of active compounds are essential for effective management. Ultimately, this approach, which includes the careful adjustment of cannabinoid concentrations alongside other bioactive components, supports a highly flexible and personalized treatment strategy. It leverages the synergistic effects of the combined ingredients to maximize therapeutic efficacy while minimizing potential side effects, providing a comprehensive solution that addresses both the physical and cognitive challenges of multiple sclerosis.

In various embodiments, the composition's cannabinoid concentrations can be adjusted to optimize therapeutic potency and enhance patient response, enabling precise fine-tuning based on the specific condition being treated, patient tolerance, and desired clinical outcomes. By varying the levels of CBD, CBG, and THC, the formulation can be tailored to support neuroprotective effects and promote the health of the myelin sheath, which is particularly advantageous for managing conditions such as multiple sclerosis, where demyelination is a key pathological feature. Personalization of the cannabinoid ratios provides a targeted approach that allows the treatment to be customized to address different aspects of the disease.

For instance, increasing the proportion of CBD may enhance the activation of the 5-HT1A receptors, contributing to improved mood regulation and reduced anxiety, which are common issues in patients with neurodegenerative disorders. The flexibility in adjusting cannabinoid ratios also enables the formulation to target specific neurotransmitter pathways and inflammatory processes, thereby optimizing the therapeutic efficacy while minimizing potential adverse effects. In cases of more advanced or severe multiple sclerosis, it may be necessary to increase the cannabinoid concentrations to achieve adequate symptom relief and mitigate the risk of complications associated with the progression of the disease. This adaptive formulation strategy provides a comprehensive and patient-specific approach, allowing for the precise calibration of the active ingredients to meet the evolving needs of the patient and maximize the clinical benefits of the therapy.

This approach enhances symptom relief, mitigates inflammation, and bolsters central nervous system health by promoting neuronal equilibrium and curbing the progression of multiple sclerosis. The use of a combination of cannabinoids such as CBD, CBG, and THC in carefully selected ratios, alongside essential vitamins and herbal extracts, reduces the likelihood of adverse effects typically associated with higher doses of single cannabinoids, thereby improving the tolerability and safety of the treatment. The strategic inclusion of multiple cannabinoids allows the formulation to harness the distinct pharmacological properties of each compound while also leveraging their synergistic interactions. In various formulations, the composition is crafted with precise concentrations of cannabinoids, meticulously balanced to maximize therapeutic efficacy.

The choice of CBD, CBG, and THC isolates is deliberate, as each cannabinoid contributes unique anti-inflammatory, neuroprotective, and immunomodulatory effects. This careful selection and balancing enhance the overall impact of the therapy, providing a multifaceted approach that targets different pathological aspects of multiple sclerosis while maintaining a high level of patient safety and comfort. The combined action of these cannabinoids supports the reduction of neuroinflammation, promotes myelin sheath integrity, and aids in the management of both physical and cognitive symptoms, offering a comprehensive and adaptable treatment option for individuals with varying stages of the disease.

In one illustrative embodiment, the composition formulated for the treatment of multiple sclerosis includes 100 mg/ml of CBD isolate, 50 mg/mL of CBG isolate, and 2.5 mg/mL of THC isolate per dose. This specific ratio of cannabinoids is designed to provide a robust therapeutic effect by targeting key pathways involved in neuroinflammation, pain modulation, and neuronal protection. However, additional embodiments may feature adjustments to these concentrations, allowing for greater flexibility in tailoring the formulation to meet the unique needs of individual patients. Such modifications enable healthcare providers to fine-tune the balance of cannabinoids based on therapeutic goals, patient response, and overall tolerability.

For example, increasing the concentration of CBD may enhance its anti-inflammatory and anxiolytic effects, while a higher proportion of CBG can be beneficial for its neuroprotective and antioxidant properties. Conversely, reducing THC levels may be advisable for patients who are sensitive to its psychoactive effects, thus minimizing potential side effects. This adaptability ensures that the composition can be personalized to optimize efficacy across a broad spectrum of MS symptoms, while also addressing the variations in patient tolerance and disease progression. By allowing precise adjustments to the cannabinoid content, the formulation supports a targeted and patient-centric approach to therapy, enhancing clinical outcomes and minimizing the risks associated with traditional, one-size-fits-all treatments.

A slight decrease in cannabinoid concentration can be advantageous for patients who demonstrate positive therapeutic responses but encounter mild side effects at higher doses. This adjustment allows for effective management of symptoms while minimizing the overall cannabinoid load, helping patients develop a more sustainable tolerance over extended treatment periods, typically ranging from three months or longer. By lowering the dosage, the risk of adverse effects is reduced, enhancing patient comfort and adherence to the regimen, particularly in long-term therapy scenarios. Conversely, an increase in cannabinoid concentration may be warranted for patients requiring more robust symptom control, especially in cases where the disease has advanced beyond the relief provided by standard dosages.

This escalation in dosage can enhance the therapeutic impact without necessitating a shift to significantly higher concentrations, thus balancing efficacy with safety. The flexibility to fine-tune the cannabinoid levels within the formulation provides a tailored approach, ensuring that the treatment remains effective across varying stages of disease progression while accommodating individual patient needs and minimizing potential side effects.

An increased concentration of cannabinoids is specifically targeted for patients experiencing chronic symptoms of multiple sclerosis, including persistent muscle tension, stiffness or spasticity, severe fatigue, depression, and sleep disturbances. In these cases, elevating the levels of CBD can significantly enhance its anti-inflammatory and neuroprotective properties, offering more effective symptom relief and improving overall neurological health. In other embodiments, the ratio of cannabinoids within the formulation can be adjusted within the defined concentration ranges to better align with individual patient needs or specific therapeutic objectives. This flexibility allows for the composition to be tailored, providing targeted support for a broad spectrum of symptoms associated with multiple sclerosis.

Additionally, the composition may be enhanced by incorporating other phytotherapeutic agents, such as herbal extracts and essential vitamins, which have shown efficacy in treating neurodegenerative conditions. For instance, the inclusion of neuroprotective herbal extracts like Hericium erinaceus, combined with vitamins such as Vitamin D, can further amplify the therapeutic benefits, addressing both the neuroinflammatory processes and the overall immune dysregulation seen in MS. In further embodiments, the cannabinoid ratios may be fine-tuned to reflect the specific severity and presentation of the patient's symptoms, allowing the formulation to be adapted for different stages of the disease. This customizable framework provides a versatile and patient-centric approach, enabling healthcare providers to adjust the treatment based on evolving clinical needs and ensuring that the composition remains effective across various manifestations of multiple sclerosis.

In another embodiment, the composition is enhanced by incorporating key components that work synergistically to provide a broad spectrum of therapeutic benefits. These include cannabinoids, which offer robust anti-inflammatory and neuroprotective effects; Hericium erinaceus (Lion's Mane extract), known for its potent anti-inflammatory and antioxidant properties, which help alleviate stress and promote overall neurological health; and Vitamin D, a critical cofactor in cellular processes and widely studied for its role in mitigating neurodegenerative conditions. The inclusion of these components is designed to provide comprehensive neuronal support, targeting the multiple pathophysiological pathways involved in multiple sclerosis (MS).

By carefully adjusting the ratios of cannabinoids and integrating these herbal extracts and vitamins, the formulation is tailored to address the complex and multifaceted nature of MS. The cannabinoids help modulate the endocannabinoid system and reduce neuroinflammation, while Lion's Mane supports neuroregeneration and enhances the integrity of neural structures. Vitamin D contributes to immune regulation and myelin health, which are essential for maintaining the function of the central nervous system. This integrated approach not only provides effective symptom relief but also offers sustained support for neurological function, helping to stabilize disease progression.

The adaptability of the formulation allows it to be finely tuned to the specific needs of individual patients, considering factors such as symptom severity, disease subtype, and patient tolerance. This flexibility makes it possible to deliver a personalized therapeutic solution that can be adjusted as the patient's condition evolves, offering a comprehensive and tailored approach to managing various forms of MS. The result is a formulation that addresses both the immediate symptoms and the underlying pathophysiology of the disease, supporting long-term neurological health and enhancing the overall quality of life for patients.

Designing therapeutic formulations for complex conditions like multiple sclerosis requires the inclusion of a diverse array of bioactive components that interact synergistically to address the multifaceted nature of the disease. The ability to adjust these components across a wide range of concentrations allows for the creation of highly adaptable and effective treatments that can be tailored to meet the specific needs of individual patients. By carefully modulating the concentrations of each active ingredient, the formulation maximizes therapeutic potential, targeting different aspects of the disease and accommodating variations in patient response and tolerance.

The flexibility to modify the composition within specific concentration ranges is key to customizing the treatment for optimal efficacy. For instance, cannabinoids such as CBD, CBG, and THC can be included in concentrations ranging from 0.25% THC to 10% CBD, with CBG typically comprising about 5%. This range allows the formulation to be adapted based on the desired therapeutic effects, such as enhanced anti-inflammatory action from CBD or immune modulation from CBG, while keeping THC levels at a minimum to reduce potential psychoactive side effects.

In a similar fashion, herbal extracts like Hericium erinaceus (Lion's Mane) can be incorporated at concentrations between 20% and 30%, offering substantial neuroprotective benefits. The high levels of bioactive compounds in Lion's Mane, such as hericenones and erinacines, support nerve growth and protect against oxidative stress, which are crucial for addressing the neurological damage and cognitive symptoms often associated with multiple sclerosis. By including Lion's Mane at these effective concentrations, the formulation enhances neural repair mechanisms and reduces inflammation, contributing to improved overall neurological health.

This approach, which allows for the precise adjustment of cannabinoids, herbal extracts, and other active ingredients, creates a customizable framework that can be tailored to the unique symptom profile and disease progression of each patient. The result is a formulation that not only offers comprehensive symptom relief but also provides ongoing support for the central nervous system, helping to manage the chronic nature of multiple sclerosis and enhance the patient's quality of life.

Lion's Mane, known scientifically as Hericium erinaceus, is a widely recognized herbal extract with significant therapeutic potential in managing multiple sclerosis (MS). This medicinal mushroom has become a well-established candidate for supporting brain and nerve health, largely due to its ability to stimulate the production of nerve growth factor (NGF), a protein critical for the maintenance, survival, and regeneration of neurons. By promoting NGF synthesis, Lion's Mane aids in the repair of damaged nerve cells and enhances neural connectivity, which is particularly beneficial for MS patients experiencing demyelination and neuronal loss.

In addition to its role in inducing NGF, Lion's Mane exhibits powerful anti-inflammatory properties, which help modulate the immune response and reduce the chronic neuroinflammation associated with MS. Its antioxidative capacity further protects neurons from oxidative stress, a key factor contributing to the progressive degeneration of nerve cells in MS. By scavenging free radicals and reducing oxidative damage, Lion's Mane supports the integrity of neural tissues and aids in preventing apoptosis, thereby preserving the structure and function of the central nervous system.

The neuroprotective effects of Lion's Mane are primarily attributed to its bioactive compounds, erinacines and hericenones, which have been extensively studied for their ability to promote neurogenesis and enhance cognitive function. Research has demonstrated that these compounds not only stimulate the production of NGF but also play a role in regulating neuroinflammatory pathways and reducing the cytotoxic effects of oxidative stress. Through these mechanisms, Lion's Mane helps stabilize neuronal health, supports myelination, and contributes to improved neurological outcomes in patients with multiple sclerosis.

The therapeutic effects of Lion's Mane extract are rooted in its strong anti-inflammatory, neuroprotective, and antioxidant properties, which effectively address the underlying physiological imbalances associated with multiple sclerosis (MS). When included in formulations at concentrations ranging from 20% to 30%, Lion's Mane serves as a key component in stabilizing the degenerative processes of the disease. Its potent anti-inflammatory action is especially valuable, given the central role of chronic inflammation in exacerbating MS severity. By modulating immune responses and downregulating pro-inflammatory cytokines, Lion's Mane helps to mitigate the neuroinflammation that contributes to ongoing myelin damage and neuronal loss.

Moreover, the neuroprotective properties of Lion's Mane are crucial in counteracting the progressive neuronal degeneration typical of MS. The bioactive compounds found in Lion's Mane, such as erinacines and hericenones, promote nerve growth factor (NGF) production, enhancing neurogenesis and supporting the repair and maintenance of neural tissues. These compounds help to protect neurons from oxidative stress-induced damage by neutralizing free radicals, thereby preserving cellular integrity and function. Additionally, the extract's capacity to reduce systemic inflammation contributes to the stabilization of neurobiological pathways linked to stress responses and neurological damage, providing a calming effect that helps alleviate MS symptoms.

By addressing both inflammation and oxidative stress, Lion's Mane not only aids in reducing neurodegeneration but also supports overall neurological health, which is particularly beneficial for patients who may not respond adequately to standard MS treatments. This holistic approach contributes to the management of a broad spectrum of MS symptoms, enhancing patient outcomes and quality of life by providing a complementary and integrative option to conventional therapies.

An imbalance between free radicals and antioxidants leads to oxidative stress, a harmful process that results in cellular damage and can significantly impair neural function. Oxidative stress is a critical factor in the progression of neurodegenerative diseases like multiple sclerosis (MS), where it exacerbates inflammation and contributes to the degradation of the myelin sheath. To counteract this, the inclusion of vitamins, particularly Vitamin D, plays a pivotal role in supporting the immune system and neurological health. Vitamin D's metabolism occurs within the central nervous system (CNS), where it actively participates in the myelination process, aiding in the repair and maintenance of nerve fibers. Adequate levels of Vitamin D are essential for modulating immune responses, as its deficiency has been linked to an increased risk of autoimmune disorders, including MS.

Deficiency in Vitamin D, as well as cannabinoids such as CBD and THC isolates, has been identified as a risk factor not only for MS but also for cardiovascular disease, cognitive decline, cancer, and mood disorders like depression and anxiety. The lack of sufficient Vitamin D impairs the regulation of immune defense cells, shifting their activity towards a harmful autoimmune response rather than protective immunity. This deregulation is a contributing factor in the onset and exacerbation of MS symptoms, making Vitamin D supplementation a key component in comprehensive treatment strategies. Recent studies underscore the importance of Vitamin D as a cofactor in the management of MS, highlighting its role in reducing inflammation, promoting remyelination, and enhancing the overall immune response.

The ability to adjust the proportions of cannabinoids, herbal extracts like Lion's Mane, and Vitamin D within the formulation offers a versatile and patient-specific therapeutic approach. This flexibility allows the treatment to be tailored to the unique physiological needs of each patient, optimizing the balance of neuroprotective, anti-inflammatory, and antioxidant effects. By customizing these critical components, the formulation can effectively address a wide range of symptoms and pathophysiological mechanisms in MS, providing a personalized and adaptable therapy that enhances clinical outcomes while reducing the risk of adverse effects.

In a preferred embodiment, the composition includes 10% CBD, 5% CBG, and 0.25% THC isolate, a ratio specifically designed to provide a balanced therapeutic effect while minimizing potential psychoactive side effects. The inclusion of a higher CBD concentration leverages its anti-inflammatory and neuroprotective properties, while CBG contributes to immune modulation and neural support, and the low concentration of THC helps manage spasticity and pain without causing significant psychoactivity. In another embodiment, the composition features a 25% concentration of herbal extracts, such as Hericium erinaceus (Lion's Mane), which enhances the neuroprotective and regenerative capabilities of the formulation. The extract's high concentration maximizes its impact on neural health, promoting nerve growth factor (NGF) production and reducing oxidative stress.

Additionally, in other preferred embodiments, Vitamin D is incorporated at a concentration of 0.013%, which is carefully selected to support immune regulation and aid in the remyelination process critical for patients with multiple sclerosis. Vitamin D's presence enhances the overall anti-inflammatory effect of the formulation and contributes to improved myelin health, potentially reducing the progression of neurological symptoms.

These embodiments provide a flexible framework for therapeutic design, enabling the formulation to be adjusted based on the severity of the patient's condition and the specific motor, cognitive, or sensory symptoms being targeted. The customizable nature of the composition allows healthcare providers to tailor the treatment to individual patient needs, optimizing efficacy and improving the overall quality of life for those with multiple sclerosis. By varying the cannabinoid ratios, herbal extract concentrations, and vitamin levels, the formulation offers a versatile approach that can be adapted as the disease progresses or as patient response evolves, ensuring a personalized and effective therapeutic strategy.

Excipients or carriers play a critical role in the formulation of pharmaceutical compositions by enhancing stability, improving bioavailability, and increasing patient acceptability, all of which are essential for maintaining the efficacy and shelf life of the product. The choice of excipients is carefully tailored to support the uniform distribution of active cannabinoids and other bioactive ingredients, ensuring that the therapeutic compounds are delivered effectively to target tissues while preserving the integrity and homogeneity of the mixture.

In this particular formulation, the excipients may include a variety of functional agents, each selected to perform specific roles that contribute to the overall quality and effectiveness of the composition. Solubilizers are employed to increase the solubility of hydrophobic cannabinoids like CBD, CBG, and THC, allowing these compounds to dissolve more readily in the formulation and thus enhancing their absorption upon administration. Emulsifiers, such as Polysorbate 80 and Sorbitan 20, are included to stabilize the mixture, facilitating the creation of a uniform dispersion of cannabinoids throughout the aqueous and lipid phases of the formulation. These emulsifying agents prevent phase separation, ensuring that each dose delivers consistent levels of active ingredients.

Stabilizers and antioxidants are incorporated to protect the formulation from degradation due to environmental factors such as light, heat, and oxygen. Antioxidants like tocopherols (Vitamin E) prevent oxidative damage to cannabinoids, maintaining their potency and extending the product's shelf life. Lipidic agents, such as medium-chain triglycerides (MCTs), act as solvents for lipophilic cannabinoids, enhancing their solubility and bioavailability while also contributing to the overall stability of the formulation.

Flavoring agents may be added to improve the palatability of the product, particularly in oral and sublingual formulations, increasing patient adherence by masking the bitter taste of cannabinoids. Aqueous agents, including purified water and glycerin, serve as carriers that facilitate the uniform dispersion of other ingredients and help achieve the desired viscosity and consistency of the formulation. Preservatives, such as methylparaben and propylparaben, are included to inhibit microbial growth, further extending the shelf life and ensuring the safety of the product during storage.

Together, these carefully selected excipients work synergistically to support the delivery, stability, and patient tolerability of the formulation. By optimizing the physical and chemical properties of the composition, they ensure that the cannabinoids and other active ingredients maintain their therapeutic efficacy throughout the product's lifecycle, providing a reliable and effective treatment option for patients with multiple sclerosis.

For example, medium-chain triglycerides (MCTs) serve as a key lipidic agent in this formulation, acting as an effective solvent for hydrophobic cannabinoids like CBD and THC. MCTs help keep these lipophilic compounds dissolved in the mixture, enhancing their solubility and ensuring they remain in a readily absorbable form upon administration. By maintaining the cannabinoids in a lipophilic environment, MCTs significantly boost bioavailability, allowing the active ingredients to be absorbed more efficiently through biological membranes. This increased solubility optimizes the delivery of cannabinoids, ensuring that therapeutic levels are achieved quickly and maintained effectively.

In addition, solubilizers and emulsifiers such as Sorbitan 20 and Polysorbate 80 are included to facilitate the uniform dispersion of cannabinoids and vitamins throughout the formulation. These agents play a crucial role in stabilizing the mixture, preventing phase separation, and ensuring that the active ingredients are evenly distributed in both the aqueous and lipid phases. By creating a stable emulsion, Sorbitan 20 and Polysorbate 80 help maintain consistent dosing, which is essential for the reliable delivery of therapeutic compounds. This consistency is particularly important in formulations designed for chronic conditions like multiple sclerosis, where precise dosing and predictable absorption are critical for effective symptom management and patient adherence.

These selected excipients work synergistically to ensure safety, efficacy, and optimal therapeutic outcomes, ultimately enhancing patient adherence. Beyond oral formulations, excipients can be tailored for other administration routes, including parenteral, sublingual, or topical, to meet individual patient needs and therapeutic objectives. For instance, flavoring or sweetening agents such as grape flavor can improve the palatability of oral formulations, facilitating patient adherence to therapy. This is particularly important in sublingual formulations designed for immediate therapeutic effects, as they rapidly address symptoms of multiple sclerosis.

In pharmaceutical formulations, especially those administered orally, a considerable part of the composition is made up of excipients. These include preservatives, antioxidants, aqueous agents, and flavoring agents, as well as stabilizers, solubilizers, emulsifiers, and lipid agents, which ensure effective delivery of the active ingredients, maintain long-term stability, and help minimize potential side effects. Typically, these excipients represent a significant percentage of the total formulation, ranging from 50% to 60%.

In formulations where active ingredients represent a minor fraction of the total composition, excipients are critical in preserving therapeutic agents' physical and chemical stability. This is especially important in oral formulations, where instability or poor solubility can compromise therapeutic efficacy and increase the risk of adverse reactions. Therefore, maintaining an optimal excipient concentration is essential for effective administration, ensuring consistent bioavailability, and extending the product's shelf life. The preferred percentage of excipients is 59.65%.

In this composition, stabilizing, solubilizing, emulsifying, preservative, and antioxidant agents, both aqueous and lipidic, are present in concentrations ranging from 9% to 12%. This high concentration guarantees a homogeneous distribution of the active ingredients, optimizes bioavailability, and ensures the safety and effectiveness of the formulation for oral use.

In different formulations, these excipients may incorporate a variety of functional agents intended to improve stability, efficacy, and overall patient experience. These agents may include solubilizers, stabilizers, emulsifiers, antioxidants, and preservatives, all of which collaborate to optimize the delivery of active ingredients such as cannabinoids. Solubilizing agents are essential in formulations comprising lipophilic compounds, ensuring that these ingredients are dissolved evenly and consequently improving their bioavailability.

In formulations that incorporate cannabinoids, which are typically lipid-soluble, emulsifying agents are vital for combining these hydrophobic compounds with water-based substances. Emulsifiers, such as Polysorbate 80 and Sorbitan 20, help to stabilize the formulation by enabling the formation of a stable oil-in-water emulsion. These agents reduce the surface tension between the lipid and aqueous phases, allowing the cannabinoids to remain evenly dispersed throughout the mixture. By preventing phase separation, the emulsifiers ensure that the cannabinoids and other active ingredients are distributed uniformly, maintaining consistency in each dose.

This stable dispersion is particularly important for achieving reliable therapeutic effects, as it prevents the cannabinoids from aggregating or settling, which could lead to variability in dosing. Consistent delivery of the active compounds is essential for maintaining the intended bioavailability and therapeutic potency, especially in formulations designed for the management of chronic conditions like multiple sclerosis. The use of effective emulsifying agents enhances overall stability, improves patient compliance by ensuring predictable absorption, and ultimately contributes to the efficacy of the treatment.

Medium-chain triglycerides (MCT) function as lipidic agents that act as a solvent for hydrophobic cannabinoids. This ensures these compounds remain dissolved and easily accessible for absorption after administration. MCTs are recognized for their capacity to form homogeneous solutions, a critical property in formulations requiring uniform distribution of active compounds, such as CBD, CBG, and THC isolates, to achieve consistent dosing and therapeutic efficacy. This characteristic is especially valuable in enhancing solubility and ensuring the sustained performance of cannabinoids within the formulation.

Additionally, MCT contributes to the overall stability of the formulation by minimizing oxidation and degradation of active cannabinoids. By maintaining cannabinoids in a stable solution with appropriate viscosity and rheological properties, MCTs effectively extend product shelf life, ensuring that therapeutic potency remains consistent throughout the product's designated half-life.

MCT is typically incorporated at concentrations between 8.0% and 16%, with an optimal level of approximately 15.0%, to provide an ideal solution while supporting formulation stability. A concentration of 15.0% MCTs is regarded as optimal for maintaining formulation stability, as it ensures cannabinoids remain evenly dispersed. At this concentration, MCTs help achieve a uniform and stable formulation, ensuring that each dose delivers a consistent amount of active ingredients, thereby enhancing therapeutic reliability for patients with every administration.

In some formulations, the emulsifying system may be enhanced by incorporating additional agents such as Sorbitan esters, Egg lecithin, Polysorbates, and Poloxamers. These ingredients are carefully selected for their unique emulsifying properties and work synergistically to create a robust emulsion system that effectively stabilizes cannabinoids, which are inherently hydrophobic. The inclusion of such a comprehensive emulsifying system plays a critical role in improving the performance and longevity of the formulation, ensuring that the active cannabinoids remain dispersed uniformly throughout the mixture.

This stability is particularly crucial in pharmaceutical formulations containing cannabinoids, as their lipophilic nature makes them prone to separation and aggregation. A well-designed emulsifying system prevents this phase separation, thereby preserving the homogeneity of the formulation. This consistency is key for maintaining bioavailability, as it ensures that each dose delivers a precise and predictable amount of active ingredients, optimizing therapeutic efficacy.

Moreover, the combination of emulsifying agents enhances the formulation's resilience under various storage and handling conditions. The stability provided by agents like Poloxamers and Egg lecithin helps the emulsion withstand temperature fluctuations, physical agitation, and prolonged storage without compromising the integrity of the mixture. This is especially important for oral cannabinoid formulations, where consistent bioavailability and precise dosing are critical for achieving the desired therapeutic outcomes. By maintaining a stable emulsion, the formulation ensures effective and reliable delivery of cannabinoids across multiple doses, supporting patient adherence and enhancing overall treatment efficacy.

Emulsifying agents, such as Polysorbates (e.g., Polysorbate 80) and Sorbitans (e.g., Sorbitan 20), play an essential role in pharmaceutical formulations, particularly those incorporating cannabinoids, which are predominantly lipophilic and prone to phase separation. These emulsifiers facilitate the creation of stable, homogeneous mixtures by lowering the interfacial tension between the oil (lipid) and water phases, thereby ensuring the even dispersion of active ingredients throughout the solution. This stabilization is especially critical for oral dosage forms, where the precise and consistent delivery of cannabinoids is necessary to achieve reliable therapeutic effects. Without the inclusion of effective emulsifying agents, cannabinoids may separate from the aqueous phase, leading to inconsistent dosing, reduced bioavailability, and diminished treatment efficacy.

Polysorbate 80 and Sorbitan 20 are synthetic emulsifiers that have been specifically chosen for their strong emulsifying properties, which help stabilize oil-in-water emulsions. Polysorbate 80, a nonionic surfactant, enhances the solubility and dispersion of cannabinoids, preventing them from coalescing or settling out of the mixture. Sorbitan 20 complements this effect by acting as a stabilizing agent, supporting the formation of a stable emulsion that maintains homogeneity throughout the shelf life of the product. Together, these emulsifiers significantly improve the dispersion and absorption of cannabinoids, making them particularly valuable in oral formulations like sublingual drops, where high bioavailability and rapid onset of action are desired.

The inclusion of Polysorbate 80 and Sorbitan 20 at specific concentration ranges further optimizes the formulation's performance. In general, Polysorbate 80 is used at concentrations ranging from 7% to 8%, with a preferred concentration of 8.0%, to ensure adequate solubilization and enhanced transport of cannabinoids across cellular membranes, facilitating efficient absorption. Similarly, Sorbitan 20 is included at concentrations of 2.0% to 3.0%, with a preferred concentration of 3.0%, to provide additional stability and prevent phase separation, even under varying storage conditions.

The strategic use of these emulsifying agents at these concentration levels contributes to the overall efficacy, safety, and reliability of cannabinoid-based oral formulations. By improving solubility, preventing separation, and promoting consistent absorption, Polysorbate 80 and Sorbitan 20 help maximize the therapeutic delivery of cannabinoids, leading to faster onset of action, improved bioavailability, and enhanced patient outcomes. This ensures that the therapeutic potential of the cannabinoids is fully realized, providing a stable and effective treatment option for conditions such as multiple sclerosis, where precise dosing and consistent bioavailability are crucial for symptom management and disease control.

In addition, antioxidants aid in cannabinoid-based formulas as these compounds are susceptible to oxidative degradation when exposed to environmental conditions such as light, heat, air, and humidity. Cannabinoids like CBD, CBG, and THC possess unsaturated bonds within their molecular structure, rendering them susceptible to oxidative degradation. Oxidative stress initiates structural changes within the cannabinoids, often producing inactive or less active metabolites, thereby reducing the overall effectiveness of the formulation.

Antioxidants play a crucial role in preserving the stability of cannabinoids and protecting oxidation-sensitive excipients within the formulation. Lipid-based excipients, for instance, are prone to oxidative degradation upon exposure to oxygen, which can compromise the structural integrity and stability of the entire formulation. Including antioxidants mitigates this risk by stabilizing these vulnerable components, thereby upholding the product's overall quality and integrity. In certain embodiments, a combination of antioxidants may be employed to establish a more comprehensive protective system against oxidative stress, enhancing the longevity and efficacy of the product.

Excessive use of certain antioxidants may cause instability in the formulation or lead to undesirable interactions with other components. In contrast, insufficient amounts of antioxidants may not provide adequate protection against oxidative degradation of cannabinoids. Therefore, it is critical to achieve an optimal balance that ensures the stability and sustained efficacy of the product.

Tocopherols, also known as Vitamin E, play a crucial role as antioxidants in maintaining the stability of cannabinoid formulations. They neutralize reactive oxygen species (ROS) and free radicals responsible for oxidative degradation.

Due to their lipid-soluble properties, tocopherols effectively protect the lipid components of the formulation, including cannabinoids. Tocopherols prevent chain reactions that could otherwise degrade the entire formulation by intercepting and stabilizing free radicals in the lipid phase. In this way, tocopherols help preserve the integrity of the cannabinoids, extending the formulation's shelf life and ensuring the active ingredients retain their potency and therapeutic effectiveness.

In various embodiments, antioxidants such as vitamin E, ascorbic acid, butylated hydroxyanisole (BHA), or combinations thereof are incorporated to stabilize the formulation and protect the cannabinoids from oxidative degradation. The presence of these antioxidants is crucial for maintaining the integrity and potency of the active ingredients, as cannabinoids are susceptible to oxidation when exposed to environmental factors like light, heat, and oxygen. By preventing oxidative damage, these stabilizing agents help preserve the therapeutic efficacy of the cannabinoids and extend the shelf life of the product.

Vitamin E, in particular, is included at concentrations ranging from 0.05% to 0.10%, with a preferred concentration of 0.05%. As a lipid-soluble antioxidant, vitamin E not only shields the cannabinoids from oxidative stress but also enhances their absorption and bioavailability. It does this by supporting the stability and integrity of lipid-based carriers within the formulation, ensuring that the cannabinoids remain dissolved and readily absorbable upon administration. This dual role of vitamin E as both a protective antioxidant and an absorption enhancer makes it a valuable component in cannabinoid-based formulations, contributing to improved patient outcomes.

The inclusion of additional antioxidants like vitamin C (ascorbic acid) further complements this effect. Vitamin C acts as a water-soluble antioxidant that helps neutralize free radicals and offers additional protection against oxidative degradation. When combined with vitamin E, the synergistic action of these antioxidants provides comprehensive stability, maintaining the therapeutic potency of the cannabinoids throughout the product's shelf life. This approach helps ensure that the formulation delivers consistent, effective doses over time, making it particularly suitable for long-term treatments such as those required for managing chronic conditions like multiple sclerosis.

Preservatives such as propylparaben, methylparaben, benzyl alcohol, and potassium sorbate are essential in prolonging the shelf life of pharmaceutical products by inhibiting microbial growth. This is especially important for oral, semi-solid, and liquid formulations, which are more susceptible to bacterial, fungal, and mold contamination due to their higher moisture content.

In other embodiments, preservatives may include but are not limited to, propylparaben, methylparaben, or combinations thereof. These preservatives stabilize the formulation by protecting it from degradation, thereby preserving potency and extending shelf life. In certain embodiments, propylparaben and methylparaben are included in a concentration range from 0.1% to 0.3%. In one preferred embodiment, the concentration for these preservatives is 0.20%. In some embodiments, the methylparaben is preferred at 0.18%. In some embodiments, the propylparaben is preferred at 0.02%.

The inclusion of preservatives and antioxidant agents is crucial for preserving the stability of formulations intended for long-term storage or frequent use. Preservatives prevent microbial contamination, while antioxidants help protect the chemical integrity of the active ingredients. This is especially important in liquid and semi-solid formulations, where the risk of degradation is higher due to moisture and other reactive compounds. By incorporating these excipients, the formulation ensures safety, stability, and efficacy throughout its shelf life, offering patients a consistent and reliable treatment option.

In various embodiments, the composition may incorporate an aqueous phase vehicle, which is essential for balancing the formulation and enhancing the solubility of active ingredients consisting of glycerin and purified water. This vehicle is included at 30% and 35% concentrations, with an optimal concentration of 32.9%. It provides a medium for dissolving or dispersing other components and optimizing the viscosity and fluidity. This water-based vehicle maintains the necessary consistency for the intended application, supporting efficient absorption and distribution of active ingredients.

Likewise, an aqueous phase vehicle contributes significantly to patient comfort and tolerability. Pharmaceutical water is a biocompatible carrier that reflects the human body's hydrophilic nature. Beyond contributing to stability and solubility, the aqueous phase vehicle increases the solubility of various components and, thus, the overall efficacy of the mixture.

The use of an oil phase vehicle, ranging from 8% to 16%, is critical to the success of the formulation. It consists of Medium Chain Triglycerides; its preferred concentration is 10.0%. This component not only improves the solubility and bioavailability of the cannabinoids but also ensures the stability and uniformity of the mixture and guarantees a comfortable experience for the patient at the time of administration. By acting as a neutral and biocompatible medium, the oil phase vehicle promotes the administration of consistent and reliable doses, which enhances the efficacy and safety of cannabinoid-based treatments, particularly in oral formulations.

In certain embodiments, the composition includes active ingredients such as CBD, CBG, and THC isolates, along with Lion's Mane root extract and Vitamin D, constituting a total concentration of 40.26%. This specific blend of cannabinoids, herbal extracts, and vitamins is designed to provide a robust therapeutic effect, targeting the neuroprotective and anti-inflammatory needs of patients, particularly those with neurodegenerative conditions like multiple sclerosis.

In another embodiment, the formulation features a flexible range of ingredient concentrations to accommodate different therapeutic needs and patient profiles. Here, the cannabinoids (CBD, CBG, and THC isolates) are included in concentrations ranging from 0.25% to 20%, while Lion's Mane root extract is present at a range of 20% to 30%. Vitamin D is incorporated at a lower concentration of 0.01% to 0.02%, aimed at supporting immune function and contributing to the overall neuroprotective benefits of the formulation.

In a further preferred embodiment, the composition is precisely formulated with specific concentrations to maximize efficacy and patient tolerability. It includes 10.0% CBD isolate, 5.0% CBG isolate, and 0.25% THC isolate, combined with 25.0% Lion's Mane root extract and 0.013% Vitamin D. This precise blend offers a balanced profile of cannabinoids to address neuroinflammation and symptom relief, while the high concentration of Lion's Mane enhances neuroregeneration and cognitive support. The inclusion of Vitamin D at this specific concentration aids in the maintenance of myelin health and overall immune modulation, contributing to a comprehensive and integrative approach for managing the complex symptoms associated with multiple sclerosis.

In another embodiment, the composition additionally comprises emulsifying agents. In a further embodiment, the emulsifier agents range from 2.0% to 10.0%. In a preferred embodiment, the composition comprises emulsifier agents such as Polysorbate 80 and Sorbitan 20. In a further preferred embodiment, the composition comprises Polysorbate 80 at 8.0% and Sorbitan 20 at 3.0%.

This pharmaceutical composition is designed to offer a range of administration routes and is available in various dosage forms, providing a flexible platform for delivering cannabinoids and other active ingredients. This approach enhances therapeutic efficacy while accommodating individual patients' unique needs and preferences. The ability to tailor the formulation for different routes of administration expands its versatility in treating a variety of medical conditions, allowing for adjustments in dosage, onset of action, and overall patient comfort.

In an alternative approach, the composition can be administered through various routes, including oral, sublingual, topical, inhaled, parenteral, and specialized topical methods, each offering distinct advantages based on the specific condition and the desired therapeutic outcomes. Oral administration, in forms such as tablets, capsules, or syrups, provides systemic distribution of the active ingredients, leading to a slower onset of action but prolonged effects. Conversely, the sublingual route delivers a faster response by allowing the ingredients to be absorbed directly into the bloodstream through the mucous membranes, bypassing the digestive system. Topical formulations, like creams and gels, are suited for localized treatment, offering targeted relief without widespread exposure to the active compounds.

Inhalation via aerosols or sprays enables rapid absorption of cannabinoids into the respiratory system, making it a preferred option for conditions requiring immediate relief, such as acute episodes of multiple sclerosis. Parenteral administration, including subdermal, subcutaneous, intravenous, intramuscular, and intra-articular injections, delivers the active ingredients precisely and swiftly. This method is particularly effective for conditions that need rapid intervention, such as multiple sclerosis and other neurological disorders.

This composition demonstrates remarkable versatility in its pharmaceutical forms, enabling the development of tablets, capsules, oral drops, syrups, suspensions, powders for reconstitution, elixirs, creams, emulsions, oral solutions, gummies, candies, sprays, oral topicals, mouthwashes, sublingual tablets, sublingual films, ovules, suppositories, injectable solutions, and injectable suspensions. Each dosage form presents distinct benefits related to stability, patient adherence, and the delivery profile of the active ingredients. For instance, sublingual tablets and films provide a discreet and convenient option that bypasses first-pass metabolism, while gummies and candies serve as an appealing alternative that promotes compliance, particularly for children and the elderly.

The pharmaceutical composition is specifically designed for oral administration, with a preferred dosage form being oral drops. This method of delivery is particularly effective for formulations containing cannabinoids, as it allows for rapid absorption through the oral mucosa, bypassing first-pass metabolism in the liver and enhancing the bioavailability of the active ingredients. The use of oral drops provides a convenient and precise dosing mechanism, enabling tailored treatment for conditions such as multiple sclerosis and other neurological disorders, where consistent and reliable symptom management is crucial.

Oral administration offers several advantages, particularly for cannabinoid-based therapies, by enabling the cannabinoids to exert potent anti-inflammatory, neuroprotective, and analgesic effects directly at the site of action. The sublingual route facilitates faster onset of therapeutic effects, as the cannabinoids are absorbed directly into the bloodstream, providing targeted relief while minimizing systemic exposure and reducing the risk of side effects. This method helps in delivering high concentrations of active ingredients directly to the central nervous system, addressing the neuroinflammatory and neurodegenerative processes underlying multiple sclerosis.

By employing an oral drop formulation, the composition can be easily adjusted to meet individual patient needs, offering a flexible and patient-friendly option for managing a wide range of symptoms, from chronic pain and spasticity to cognitive impairments. This approach ensures a more controlled and consistent release of cannabinoids, improving patient adherence and allowing for better overall symptom control in complex and chronic conditions like multiple sclerosis.

Bioequivalence is a crucial aspect, highlighting the flexibility and adaptability of this pharmaceutical composition across various forms and routes of administration. This principle underpins patent protection by covering not just specific embodiments but also equivalent variations that serve the same function similarly and yield the same result. For instance, a composition originally intended for oral use may have an equivalent formulation suitable for parenteral or topical administration, provided it delivers the same therapeutic effect using similar ingredients or methods.

This flexibility allows the composition to be customized to suit the needs of different patient populations and medical conditions without altering its fundamental properties. For example, a formulation initially designed as an oral drop solution could be adapted with advanced bioavailability technologies for a different oral delivery method and still be considered equivalent under this legal framework, offering the same therapeutic benefits.

The design of this pharmaceutical composition allows for a range of administration routes and dosage forms, showcasing its versatility for diverse therapeutic purposes. The concept of bioequivalence further enhances this adaptability, ensuring that even minor adjustments in delivery methods are protected under patent law.

In an embodiment, the present disclosure describes a therapeutic composition for treating multiple sclerosis. The formulation is designed to leverage the synergistic effects of cannabinoids, herbal extracts, vitamins, and emulsifying agents to provide a comprehensive and effective treatment approach. The composition includes the following components:
a. CBD, CBG, and THC in a concentration ranging from 0.25% to 30.0%: The primary active ingredients are cannabinoids, specifically cannabidiol (CBD), cannabigerol (CBG), and tetrahydrocannabinol (THC). These cannabinoids are included in concentrations ranging from 0.25% to 30.0%, allowing flexibility in tailoring the formulation based on the severity of multiple sclerosis symptoms and the specific needs of the patient. CBD is primarily incorporated for its potent anti-inflammatory and neuroprotective effects, while CBG enhances immune modulation and neural protection. THC, included at controlled concentrations, provides relief from spasticity and chronic pain without significant psychoactive effects. The broad concentration range allows for customized dosing strategies, optimizing therapeutic efficacy.
b. Herbal extract in a concentration ranging from 20.0% to 30.0%: The composition includes a herbal extract, such as Hericium erinaceus (Lion's Mane), in concentrations between 20.0% and 30.0%. This extract is chosen for its neuroregenerative properties, supporting nerve growth factor (NGF) production, enhancing cognitive function, and protecting against oxidative stress. The specified concentration range ensures that the herbal extract is present at therapeutic levels, contributing to the overall neuroprotective profile of the formulation and addressing key neurodegenerative aspects of multiple sclerosis.
c. Vitamin D in a concentration ranging from 0.01% to 0.02%: Vitamin D is incorporated as a critical cofactor in the composition, included at concentrations ranging from 0.01% to 0.02%. Even at these low levels, Vitamin D plays an essential role in immune regulation, supporting the repair and maintenance of myelin, and enhancing overall neurological health. Adequate Vitamin D levels help mitigate autoimmune responses and reduce inflammation, which are significant factors in the progression of multiple sclerosis. Its inclusion complements the effects of cannabinoids and herbal extract, providing a balanced and integrative therapeutic approach.
d. Emulsifying agents in a concentration ranging from 2.0% to 10.0%: The composition includes emulsifying agents, such as Polysorbate 80 and Sorbitan 20, at concentrations ranging from 2.0% to 10.0%. These agents are vital for creating a stable oil-in-water nanoemulsion, ensuring the uniform dispersion of the hydrophobic cannabinoids and herbal extracts throughout the aqueous phase of the formulation. Emulsifiers enhance the bioavailability of the active ingredients by increasing solubility and absorption, making the formulation suitable for oral administration. The specified range allows for adjustments in the emulsifier concentration to optimize the stability, consistency, and patient acceptability of the product.

This therapeutic composition, with its precise balance of cannabinoids, herbal extracts, vitamins, and emulsifying agents, is designed to offer targeted relief for the complex symptoms of multiple sclerosis. The formulation provides a holistic approach that addresses neuroinflammation, supports neural repair, and enhances overall neurological health, contributing to improved patient outcomes and quality of life.

In an embodiment of the disclosure, the therapeutic composition is an advanced formula developed for the treatment of multiple sclerosis, designed specifically for administration via oral drops. This targeted delivery method ensures precise dosing and rapid absorption, facilitating the efficient relief of symptoms. The formulation includes:
CBD at 10%: Selected for its robust anti-inflammatory and neuroprotective effects.
CBG at 5%: Provides immune modulation and additional neuroprotective properties.
THC at 0.25%: Included at a low dose for spasticity relief while minimizing psychoactive effects.
Hericium erinaceus extract at 25%: Chosen for its strong neuroregenerative capabilities, supporting neural growth.
Vitamin D at 0.013%: Incorporated for its role in immune regulation and myelin repair.
Polysorbate 80 at 8% and Sorbitan 20 at 3%: Used as emulsifying agents to stabilize the nanoemulsion, enhancing the solubility and bioavailability of the active ingredients.

This composition leverages a nanoemulsion delivery system, ensuring uniform dispersion and improved absorption of cannabinoids and herbal extracts. By utilizing a carefully balanced blend of these components, the formulation offers comprehensive support for multiple sclerosis, targeting neuroinflammation, spasticity, cognitive issues, and immune dysfunction. The oral drops format provides a patient-friendly method of administration, optimized for consistent and effective therapeutic outcomes.

In an embodiment of the disclosure, this therapeutic composition is designed for the treatment of multiple sclerosis, utilizing a specialized blend of active ingredients and emulsifiers, formulated for administration as oral drops. The key components include:
a. CBD at 20% concentration: This high level of cannabidiol (CBD) provides potent anti-inflammatory and neuroprotective effects, targeting the chronic pain, spasticity, and neuroinflammation often experienced by multiple sclerosis patients.
b. CBG at 5% concentration: Cannabigerol (CBG) is incorporated at 5% to offer additional neuroprotective benefits and immune modulation, complementing CBD's action and supporting overall neural health.
c. THC at 0.25% concentration: Tetrahydrocannabinol (THC) is included at a low concentration of 0.25%, allowing for effective relief from spasticity and neuropathic pain, while minimizing psychoactive effects.
d. Hericium erinaceus extract at 25% concentration: The herbal extract of Hericium erinaceus (Lion's Mane) is included at 25%, chosen for its significant neuroregenerative properties, which promote nerve growth factor (NGF) production and enhance cognitive function, addressing the neurodegenerative symptoms of multiple sclerosis.
e. Vitamin D at 0.013% concentration: Vitamin D is incorporated as a critical cofactor, aiding in immune regulation and promoting the maintenance of myelin integrity, essential for reducing neuroinflammation and supporting neural repair.
f. Polysorbate 80 at 7% concentration and g. Sorbitan 20 at 2% concentration: These emulsifying agents are employed to create a stable oil-in-water nanoemulsion, enhancing the solubility and bioavailability of the cannabinoids and herbal extracts. Polysorbate 80 and Sorbitan 20 work synergistically to prevent phase separation, ensuring a homogeneous mixture and consistent dosing.

Formulated for oral drops administration: The composition is specifically prepared for delivery via oral drops, a method that enables precise, patient-friendly dosing and efficient sublingual absorption. The nanoemulsion technology used in this formulation ensures rapid onset of action and improved bioavailability of the active compounds, facilitating targeted symptom relief and comprehensive management of multiple sclerosis.

In summary, this therapeutic composition provides a balanced, integrative approach to managing the diverse symptoms of multiple sclerosis, addressing both the physical and cognitive challenges of the disease. The carefully selected ingredients and advanced formulation techniques offer a robust treatment option that enhances patient outcomes through improved bioavailability, consistent dosing, and sustained therapeutic effects.

### Treatment Protocol:

In one embodiment, the composition can be customized for various routes of administration beyond oral, depending on the patient's specific condition and desired therapeutic results. These routes include topical and transdermal formulations, offering flexible treatment options. Furthermore, the composition can be prepared in different dosage forms, such as solutions, emulsions, or nanoemulsions, to optimize bioavailability and enhance patient adherence. It can also incorporate various controlled-release mechanisms, including immediate, sustained, or delayed release, to improve the therapeutic experience. These options facilitate the gradual release of active cannabinoids such as CBD, CBG, and THC over time, thereby enhancing effectiveness and reducing the need for frequent dosing. This versatility allows healthcare providers to tailor treatments based on the severity of MS, patient preferences, and individual medical needs.

The compositions described in this disclosure may be presented in numerous pharmaceutical dosage forms, customized to improve patient compliance and optimize therapeutic efficacy in various healthcare settings. These forms include solid, semi-solid, liquid, and gaseous preparations, each precisely designed to target distinct routes of administration, ensuring adaptability in treatment and maximizing therapeutic benefits.

In one embodiment, the pharmaceutical composition comprising phytocannabinoids is designed to be compatible with multiple administration routes, accommodating various therapeutic contexts. These methods may include oral, sublingual, topical, inhalation, and parenteral administration. Each route provides treatment flexibility and precise dose control, enhancing bioavailability and ensuring that the cannabinoids reach their intended sites of action to address diverse patient needs.

In this modality, capsules, tablets, and solutions (drops) are very convenient oral formulations, especially for patients looking for an easy-to-use option to manage chronic diseases over the long term. For faster results, sublingual forms, such as tablets, solutions (drops), sprays, and soluble films, bypass gastrointestinal metabolism, allowing cannabinoids to be absorbed directly into the bloodstream. Oral and sublingual delivery methods are ideal for systemic treatments, as they offer prolonged bioavailability of the active compounds.

In one modality, topical formulations, including transdermal patches, are designed to release the active compounds into the body through the skin. Transdermal patches are particularly effective, as they offer a constant and controlled release of cannabinoids as they enter the bloodstream, making them suitable for alleviating symptoms associated with multiple sclerosis.

In a further embodiment, the use of inhalation via aerosols, inhalers, or vaporized cannabinoid products offers a convenient and fast-acting method of delivering therapeutic benefits. This route allows cannabinoids to be rapidly absorbed through the lungs, making it ideal for immediate relief of symptoms associated with the progression of multiple sclerosis, such as respiratory failure, respiratory rate disturbances, and respiratory muscle weakness. By avoiding first-pass metabolism, inhalation provides rapid delivery of cannabinoids into the bloodstream with an almost instantaneous onset of effects.

The compositions of this disclosure may be available in liquid forms and may include syrups, solutions, emulsions, or suspensions. Liquids are suitable for oral, sublingual, or topical administration and offer ease of dosing and rapid absorption, making them suitable for patients seeking rapid relief or who have difficulty swallowing solid forms.

Parenteral cannabinoid formulations, designed for administration via routes such as intradermal, intraarticular, subcutaneous, intravenous, intramuscular, or intrathecal injections, enable precise and controlled delivery, particularly in clinical environments where rapid therapeutic outcomes are essential. Intravenous formulations provide quick systemic delivery of cannabinoids, offering immediate relief for acute symptoms such as neuropathic pain, muscle spasms, tremors, tingling, itching, or burning sensations in the extremities, and limb stiffness or numbness. On the other hand, subcutaneous and intramuscular injections release cannabinoids more gradually, ensuring sustained therapeutic effects for the management of chronic conditions.

Additionally, the composition may utilize nanotechnology-driven delivery systems to enhance bioavailability and ensure more targeted absorption. Nanoparticles, nanoemulsions, or liposomes can safeguard cannabinoids from degradation and improve their ability to cross biological barriers, such as the blood-brain barrier or the skin. These advanced delivery methods enable more efficient absorption and prolonged retention of cannabinoids at therapeutic levels, leading to more effective symptom relief with fewer side effects.

The compositions described in this disclosure may be presented in liquid forms, including syrups, solutions, emulsions, or suspensions. These liquid formulations are suitable for oral, sublingual, or topical use, providing ease of administration and fast absorption. They are an ideal option for patients who require rapid relief or those who have difficulty swallowing solid dosage forms.

In another variation, the composition could be delivered in gaseous forms, such as via inhalers or aerosols, enabling rapid absorption of cannabinoids through the respiratory tract. This provides a quick and efficient means of achieving systemic effects, making it particularly effective for the fast relief of acute symptoms associated with multiple sclerosis.

In certain formulations, dosage forms may include immediate-release, sustained-release, controlled-release, or delayed-release options, providing precise control over cannabinoid bioavailability throughout the day. Immediate-release formulations offer quick relief for acute symptoms, while sustained-release versions, such as nanoparticle-based delivery systems, gradually release active ingredients over time. Controlled-release formulations maintain a consistent rate of cannabinoid release, ensuring stable levels in the body for effective management of chronic symptoms and enhanced patient adherence. Delayed-release formulations can be tailored to meet the needs of conditions requiring localized or time-sensitive treatment. These flexible release mechanisms cater to a wide range of therapeutic needs, from fast symptom relief to long-term management of multiple sclerosis-related symptoms.

### Method of Treatment:

The method for treating multiple sclerosis in a human individual comprises administering an effective concentration of the composition described in the disclosure, where the active principles include at least two cannabinoids, at least one herbal extract enriched with vitamins, and at least two emulsifying agents. The treatment method is designed to target the complex and multifaceted symptoms of multiple sclerosis, offering a comprehensive therapeutic approach that addresses both neurological and immunological aspects of the disease.

The cannabinoids, selected from CBD, CBG, and THC, work synergistically to provide potent anti-inflammatory, neuroprotective, and analgesic effects. The combination of at least two cannabinoids allows for modulation of the endocannabinoid system, reducing neuroinflammation, alleviating spasticity, and managing chronic pain, which are common symptoms experienced by MS patients. By targeting CB1 and CB2 receptors, the cannabinoids help to restore neural balance, reduce muscle rigidity, and support cognitive function, contributing to overall symptom relief.

The herbal extract, specified as Hericium erinaceus and enriched with vitamins such as Vitamin D, provides additional neuroprotective and regenerative benefits. Hericium erinaceus promotes nerve growth factor (NGF) production, supports neural repair, and reduces oxidative stress, which are critical for mitigating the neurodegenerative processes associated with multiple sclerosis. The inclusion of Vitamin D aids in regulating immune responses and enhances myelin repair, further supporting neurological health and helping to control disease progression.

The presence of at least two emulsifying agents, such as Polysorbate 80 and Sorbitan 20, ensures that the composition is delivered in a stable nanoemulsion format. These emulsifiers improve the solubility and bioavailability of the lipophilic cannabinoids, allowing for consistent absorption and efficient transport across biological membranes. The nanoemulsion enhances the distribution of the active ingredients, ensuring that therapeutic levels are maintained throughout the treatment period.

This method involves the administration of the composition in a carefully controlled dosing regimen, tailored to the severity of the patient's symptoms and overall health condition. The precise and consistent delivery of the active principles allows for targeted symptom management, reduces neuroinflammation, and supports neural regeneration. By employing a combination of cannabinoids, herbal extracts, vitamins, and emulsifiers, the method provides an integrative treatment strategy designed to alleviate the symptoms of multiple sclerosis and improve the quality of life for patients.

The method of disclosure specifies that the human subject is selected for treatment based on a confirmed diagnosis of multiple sclerosis (MS). This targeted approach ensures that the composition is administered to individuals who have been clinically identified with MS, a neurodegenerative disease characterized by chronic inflammation, demyelination, and progressive neurological impairment. By selecting patients based on a definitive MS diagnosis, the treatment method aims to address the specific pathophysiological mechanisms of the disease, including neuroinflammation, immune dysregulation, and neuronal damage.

The inclusion criteria based on diagnosis allow healthcare providers to tailor the therapeutic regimen to meet the distinct needs of MS patients, optimizing the potential benefits of the composition. The formulation, comprising cannabinoids, herbal extracts, and vitamins, is designed to mitigate the core symptoms of multiple sclerosis, such as spasticity, chronic pain, fatigue, and cognitive dysfunction. Through this patient selection process, the method ensures that the treatment is provided to those most likely to benefit from its comprehensive neuroprotective and anti-inflammatory effects, enhancing the efficacy of the intervention and contributing to better overall patient outcomes.

The method of the disclosure specifies that the individual is selected for treatment based on the diagnosis of multiple sclerosis (MS) and includes various subtypes of the disorder, such as Relapsing-Remitting Multiple Sclerosis (RRMS), Secondary Progressive Multiple Sclerosis (SPMS), Primary Progressive Multiple Sclerosis (PPMS), and Progressive-Relapsing Multiple Sclerosis (PRMS). This comprehensive approach allows the method to be adapted to treat the full spectrum of MS presentations, each of which exhibits distinct clinical features and progression patterns.

For individuals with Relapsing-Remitting MS (RRMS), characterized by episodes of new or worsening symptoms followed by periods of remission, the composition aims to reduce the frequency and severity of relapses by targeting neuroinflammation and supporting immune regulation. In cases of Secondary Progressive MS (SPMS), where the disease transitions from a relapsing course to a more progressive decline in neurological function, the method focuses on neuroprotective and regenerative effects to help slow the progression of disability.

For Primary Progressive MS (PPMS), which involves a gradual worsening of symptoms from the onset without distinct relapses, the method prioritizes sustained symptom relief and the stabilization of neurological health through controlled release and high bioavailability of the active ingredients. In treating Progressive-Relapsing MS (PRMS), characterized by a steady progression with intermittent relapses, the method combines anti-inflammatory and neuroprotective actions to manage both the ongoing progression and the acute exacerbations.

By including a broad range of MS subtypes, the treatment method provides a versatile and adaptable therapeutic strategy, tailored to the unique pathophysiological needs of each patient. The targeted use of cannabinoids, herbal extracts, and vitamins in the composition is intended to alleviate the diverse symptoms associated with these MS variants, offering a comprehensive approach that enhances patient outcomes and improves quality of life across different forms of the disease.

The method of disclosure further specifies that the individual selected for treatment is identified based on the presence of characteristic symptoms commonly associated with multiple sclerosis. These symptoms include, but are not limited to:
Fatigue: A pervasive and debilitating tiredness that is one of the most common symptoms of multiple sclerosis, often affecting daily activities and quality of life.
Vision problems: Issues such as blurred vision, double vision, or loss of vision, often resulting from inflammation of the optic nerve (optic neuritis), which is a frequent early sign of MS.
Motor difficulties: Weakness, loss of coordination, and impaired gait that arise due to damage to the nerve pathways responsible for muscle movement.
Abnormal sensations: Sensory disturbances such as numbness, tingling, burning sensations, or electric shock-like sensations (Lhermitte's sign), reflecting nerve damage in the central nervous system.
Cognitive problems: Impairments in memory, attention, and executive function, which can affect the patient's ability to process information and perform daily tasks.
Spasticity: Muscle stiffness and involuntary muscle spasms that contribute to pain, reduced mobility, and discomfort, commonly affecting the legs.
Bladder and bowel issues: Problems such as urinary urgency, incontinence, or constipation, stemming from disrupted nerve signals that control the bladder and bowel functions.
Speech disturbances: Difficulties with speech, including slurred speech (dysarthria) and challenges in articulation, which may result from damage to the brain areas involved in motor control.
Depression and Anxiety: Mood disturbances that are common in MS patients, possibly linked to both the psychological impact of the disease and changes in brain chemistry caused by inflammation and neural damage.

By identifying individuals who exhibit one or more of these symptoms, the method targets patients with a confirmed or suspected diagnosis of multiple sclerosis, ensuring that the treatment is provided to those who are most likely to benefit from the composition's neuroprotective, anti-inflammatory, and symptomatic relief properties. This approach allows for tailored treatment that directly addresses the diverse and multifaceted manifestations of MS, improving patient outcomes and enhancing quality of life.

The method of disclosure further specifies that the effective concentration of the composition is administered as a 0.5 mL dose, taken every 8 hours for a duration of 3 months. This precise dosing regimen is designed to provide consistent therapeutic levels of the active ingredients throughout the day, optimizing symptom relief and maximizing the therapeutic benefits of the formulation.

The administration of 0.5 mL every 8 hours ensures that the cannabinoids, herbal extracts, and vitamins are delivered in a steady and controlled manner, allowing for sustained bioavailability and prolonged effects. This schedule aligns with the typical pharmacokinetic profile of the active compounds, minimizing fluctuations in plasma concentration and reducing the risk of symptom recurrence. By maintaining consistent dosing intervals, the method supports effective management of the chronic symptoms associated with multiple sclerosis, such as spasticity, pain, and cognitive disturbances.

The 3-month treatment period is intended to provide sufficient time for the active ingredients to exert their full therapeutic effects, including the modulation of neuroinflammation, support for myelin repair, and enhancement of neural health. This extended duration of therapy allows for comprehensive symptom management and contributes to improved patient outcomes by addressing both the acute and progressive aspects of multiple sclerosis.

The method of the disclosure specifies that the administration of the effective concentration of the composition is utilized as a monotherapy for treating multiple sclerosis. In this embodiment, the composition is designed to serve as the sole therapeutic intervention, without the need for additional pharmaceutical agents. By leveraging the combined effects of cannabinoids, herbal extracts, and vitamins, the formulation aims to provide comprehensive symptom relief and address the underlying pathophysiological processes of multiple sclerosis.

The use of the composition as a monotherapy offers a simplified and patient-friendly treatment regimen, reducing the complexity and potential side effects associated with polypharmacy. The cannabinoids, including CBD, CBG, and THC, target multiple pathways involved in the disease, offering potent anti-inflammatory, neuroprotective, and analgesic effects. These compounds modulate the endocannabinoid system, helping to alleviate chronic pain, reduce spasticity, and improve overall neurological function.

The inclusion of Hericium erinaceus (Lion's Mane extract) and Vitamin D enhances the neuroprotective profile of the monotherapy, supporting neural repair, promoting nerve growth factor (NGF) production, and regulating immune responses. This combination addresses the neuroinflammatory and neurodegenerative components of multiple sclerosis, contributing to a holistic treatment approach.

As a monotherapy, the method provides a streamlined and integrative solution for patients, minimizing the risk of drug-drug interactions and reducing the treatment burden. The formulation's design aims to offer a balanced and effective therapeutic option, targeting the diverse symptoms of multiple sclerosis while improving patient adherence and optimizing long-term outcomes.

The method of the disclosure specifies that the treatment composition is designed to alleviate a wide range of symptoms commonly associated with multiple sclerosis, including fatigue, vision problems, motor difficulties, abnormal sensations, cognitive problems, spasticity, bladder and bowel issues, speech disturbances, depression, and anxiety. By targeting these diverse symptoms, the composition provides a comprehensive therapeutic approach that addresses both the physical and psychological manifestations of the disease.

The cannabinoids (CBD, CBG, and THC) play a key role in alleviating fatigue, motor difficulties, and spasticity by interacting with the endocannabinoid system to modulate pain pathways, reduce muscle stiffness, and improve overall mobility. CBD's strong anti-inflammatory and neuroprotective properties help to mitigate chronic fatigue by reducing systemic inflammation and supporting neural function. The presence of CBG enhances neuroprotective effects, aiding in muscle coordination and reducing motor impairments, while THC at a low concentration contributes to muscle relaxation, helping alleviate spasticity.

The inclusion of Hericium erinaceus (Lion's Mane extract) is critical for addressing cognitive problems and abnormal sensations, such as numbness and tingling. This neuroprotective extract promotes the synthesis of nerve growth factor (NGF), supporting neural regeneration and enhancing cognitive function, which can be impaired in MS patients. Additionally, the extract's antioxidant properties help protect nerve cells from oxidative damage, reducing sensory disturbances and promoting neural health.

Vitamin D, as part of the composition, contributes to the management of vision problems, bladder and bowel issues, and speech disturbances by supporting myelin repair and regulating immune responses. Its role in enhancing myelin health helps improve nerve signal transmission, addressing the neurological dysfunctions that underlie these symptoms.

Finally, the cannabinoids and herbal extracts work synergistically to alleviate depression and anxiety, common mood disorders in MS patients. The anxiolytic effects of CBD, combined with the neuroregenerative properties of Lion's Mane and the mood-stabilizing influence of Vitamin D, help modulate neurotransmitter levels, supporting emotional stability and mental well-being.

By addressing this comprehensive range of symptoms, the treatment method provides a holistic and integrative approach, enhancing the overall quality of life for individuals with multiple sclerosis. The formulation's ability to target multiple symptom domains ensures broad-spectrum relief, making it a versatile and effective option for patients experiencing the diverse challenges associated with the disease.

The method of disclosure further comprises the step of identifying individuals suitable for treatment based on a comprehensive diagnostic approach. This process involves an evaluation that includes the patient's medical history, detailed physical examination, and a review of laboratory values with a focus on immunological antibody profiles. By analyzing specific immunological markers, such as the presence of oligoclonal bands in cerebrospinal fluid or elevated levels of certain autoantibodies, healthcare providers can gain critical insights into the immune dysregulation associated with multiple sclerosis.

Additionally, the identification process includes the use of a symptom diary, where patients document their neurological and immunological symptoms over time, allowing for the tracking of patterns, relapses, and progression. This patient-reported information provides valuable context and supports the clinical assessment, helping to distinguish between different MS subtypes and track disease activity.

Further neurological evaluation involves the use of standardized assessment tools, such as the Expanded Disability Status Scale (EDSS). The EDSS is a widely accepted scoring system used to quantify the level of disability in MS patients, providing a measure of neurological function across several domains, including motor skills, sensory function, and coordination. The EDSS score helps classify the severity and progression of the disease, enabling clinicians to tailor the treatment plan accordingly.

By employing this multi-faceted diagnostic approach, the method ensures accurate identification of individuals who are most likely to benefit from the composition. This thorough evaluation process aids in selecting patients with confirmed multiple sclerosis, optimizing the treatment's effectiveness, and supporting a personalized therapeutic strategy that addresses the specific needs and progression of the disease.

The method of treatment is designed as a sublingual solution, tailored for direct administration under the tongue. Each drop of the formulation contains 100 mg/mL of CBD isolate, 50 mg/mL of CBG isolate, 2.5 mg/ml of THC isolate, 250 mg/ml of Hericium erinaceus extract, and 0.13 mg/mL of Vitamin D. This sublingual delivery method allows for the efficient absorption of bioactive compounds through the oral mucosa, facilitating rapid entry into the bloodstream and offering a potent therapeutic option for managing multiple sclerosis.

By utilizing sublingual administration, the treatment bypasses the gastrointestinal tract and first-pass metabolism, significantly enhancing the bioavailability of the active ingredients. This targeted delivery method ensures that the cannabinoids, herbal extracts, and vitamins reach the central nervous system more effectively, maximizing their neuroprotective and anti-inflammatory effects. The high concentration of CBD provides substantial anti-inflammatory action, while CBG contributes additional neuroprotective benefits and immune modulation. THC at a lower concentration aid in the relief of spasticity and pain without producing pronounced psychoactive effects.

Hericium erinaceus extract (Lion's Mane) is included at a therapeutic dose to support neural regeneration and cognitive function, leveraging its known neuroprotective properties. The addition of Vitamin D helps regulate immune responses and promotes myelin health, addressing critical aspects of multiple sclerosis pathology. This method of treatment, with its precise formulation and efficient delivery system, offers a comprehensive and integrative approach for alleviating the diverse symptoms associated with multiple sclerosis, providing patients with a reliable and effective therapeutic option.

The method of treatment involves sublingual drops specifically formulated for the management of multiple sclerosis, designed to address the complex neurochemical and immunological pathways involved in the disease. The sublingual administration targets the rapid absorption of active ingredients, allowing them to modulate key neurotransmitter systems, including serotonin, dopamine, and GABA. By influencing these neurotransmitters, the formulation helps regulate mood, alleviate anxiety, and enhance cognitive function, offering broad neurological benefits to patients suffering from MS.

In addition to neurotransmitter modulation, the cannabinoids in the formulation act centrally through CB1 receptor activation, which plays a critical role in regulating motor and nociceptive pathways. This mechanism helps alleviate symptoms such as muscle spasticity, chronic pain, and motor dysfunction commonly experienced by MS patients. By interacting with CB1 receptors, the cannabinoids help restore neural balance and improve motor control, contributing to enhanced mobility and reduced discomfort.

The treatment method also triggers anti-inflammatory responses via the induction of interleukin-1 receptor antagonist (IL-1ra), a cytokine known for its potent anti-inflammatory effects. By upregulating IL-1ra, the formulation effectively reduces neuroinflammation, a key driver of MS progression. This action helps rebalance the immune response, mitigate oxidative stress, and protect neuronal health. The combination of neurotransmitter regulation and targeted anti-inflammatory pathways provides a comprehensive approach, addressing both the physical and psychological aspects of multiple sclerosis, and contributing to improved patient outcomes.

In this embodiment, these concentrations are designed to provide effective relief of multiple sclerosis symptoms such as cognitive changes, spasticity, chronic pain, bladder and bowel dysfunction, fatigue, numbness and tingling, muscle weakness, vision problems, coordination and balance issues, and emotional changes.

In additional embodiments, this formulation exerts its effects via modulation of the endocannabinoid system (ECS), specifically through interaction with cannabinoid receptors CB1 and CB2 expressed within the central nervous system. These receptors play a critical role in mediating symptom evolution and severity in multiple sclerosis. The composition comprises active phytocannabinoids such as CBD, CBG, THC, essential vitamins, and selected herbal extracts, synergistically targeting ECS pathways to enhance therapeutic efficacy.

In other embodiments, CBD, along with cannabigerol (CBG), both at the proposed doses, can increase the expression of dopaminergic receptors 2 and 4 in NSC-34 cells while simultaneously inhibiting the expression of monoamine transporter 2, thereby reducing dopamine uptake. This may also play a role in synaptic plasticity.

In one embodiment, the treatment is designed for adult patients of any gender who show symptoms of multiple sclerosis. The formulation is intended for individuals aged 18 to 65 years, covering a broad patient demographic that includes both early and advanced stages of the disease. This age range allows for the effective management of symptoms commonly associated with multiple sclerosis, such as spasticity, chronic pain, cognitive impairment, and mood disturbances, while also addressing the needs of younger adults experiencing early onset as well as older patients dealing with progressive forms of the disease. The versatility of the formulation, combined with its targeted therapeutic effects, makes it suitable for a wide range of adult patients seeking relief from the complex and multifaceted symptoms of multiple sclerosis.

In one variation, the composition utilizing a nanoplatform delivery system with phytocannabinoids is administered as oral drops for sublingual use, divided into three daily doses spaced 8 hours apart. The treatment is intended to last for a duration of 3 months, with adjustments based on ongoing symptom monitoring and patient response. This structured dosing schedule ensures consistent therapeutic levels of cannabinoids, maximizing their efficacy while providing sustained relief from symptoms associated with multiple sclerosis.

In another variation, the method of treatment involves administering a dose containing specific active ingredient concentrations: 10.0% CBD, 5.0% CBG, and 0.25% THC. This precise cannabinoid ratio is designed to offer a balanced therapeutic effect, addressing both neuroinflammation and symptom relief. The dosing regimen consists of taking 0.5 mL per dose, equating to 40.26 mg of active ingredients per mL, every 8 hours for a period of 3 months. This approach ensures that patients receive a consistent and effective dose throughout the day, promoting optimal bioavailability and therapeutic outcomes while minimizing potential side effects. The regular administration schedule supports steady absorption and helps maintain the desired levels of cannabinoids in the bloodstream, contributing to improved symptom control and overall patient well-being.

In other variations, the treatment approach may be adjusted based on the specific dosing regimen and schedule, which are critical factors for achieving a balance between therapeutic effectiveness and patient tolerability. For certain patients, a single-dose treatment strategy may be employed, where a higher concentration of phytocannabinoids or other active compounds is administered to provide rapid relief from acute symptoms of multiple sclerosis. This approach can be particularly beneficial during flare-ups or periods of exacerbated symptoms, allowing for quick intervention and symptomatic relief.

Alternatively, an adaptive treatment plan may be implemented, tailored to accommodate changes in the patient's symptoms, disease progression, or overall health status. In this scenario, the dosing regimen can be modified dynamically, with adjustments made to the dosage strength, frequency, or the combination of active ingredients based on ongoing patient assessments. For instance, if a patient shows improvement in symptoms or experiences reduced inflammation, the dosage may be gradually reduced to maintain symptom control while minimizing the risk of side effects. Conversely, if the patient's condition worsens or new symptoms emerge, the treatment plan can be intensified, increasing the dosage or incorporating additional therapies to address the new clinical needs.

This adaptive, patient-centered approach ensures a personalized treatment experience that is responsive to the individual's evolving condition. By continuously monitoring the patient's response and making necessary adjustments, healthcare providers can optimize the safety and effectiveness of the therapy throughout the course of treatment. This strategy not only enhances the overall efficacy of the regimen but also supports better long-term management of multiple sclerosis, contributing to improved patient outcomes and quality of life.

### Manufacturing Method:

In certain embodiments, the fabrication of the formulation adheres strictly to Good Manufacturing Practices (GMP) for pharmaceutical products, with a focus on liquid products, specifically oral drops. This ensures the highest standards of safety, quality, and consistency throughout the production process. In alternative embodiments, the formulation is purposefully designed in a liquid drop format, allowing for convenient administration and precise dosing. The active ingredients undergo ultrasonic cavitation, a process that generates nanoemulsions aimed at enhancing the bioavailability and therapeutic efficacy of the formulation.

Nanoemulsions are preferred for their superior functional properties, providing increased stability, enhanced optical clarity, and improved absorption compared to traditional emulsions.

The nanoemulsion system operates on a nanoscale level, utilizing ultrasonic or acoustic cavitation to achieve the desired particle size reduction. During this process, intense shear forces are generated, supplying the energy required to break down large droplets into nanometer-sized particles. The result is a stable, uniform emulsion with droplets typically ranging from 0.1 to 200 nm in diameter. This reduction to nanometer-sized particles increases the surface area of the active ingredients, significantly enhancing their solubility and bioavailability, which is particularly beneficial for hydrophobic compounds like cannabinoids.

In another embodiment, high-pressure homogenization is employed as an alternative method to achieve the necessary shear forces for nanoemulsion formation. This technique uses extreme pressure to reduce the size of droplets and ensure a homogeneous blend of two or more liquid phases, resulting in a nanometer or submicron-sized emulsion. High-pressure homogenization effectively disperses the active ingredients throughout the mixture, creating a stable emulsion that enhances the delivery of bioactive compounds.

In one preferred embodiment, the resulting emulsion is a biphasic system where one phase (oil) is distributed within the other (water) as fine droplets measuring between 0.1 and 200 nm in diameter. While such systems are thermodynamically unstable due to the natural tendency of the dispersed droplets to coalesce, stability is achieved by incorporating emulsifying agents. These agents, commonly referred to as emulsifiers or emulgents, play a key role in maintaining the integrity of the nanoemulsion, preventing phase separation and ensuring consistent performance and efficacy of the formulation. This stabilized nanoemulsion system offers enhanced delivery and absorption of the active ingredients, supporting a more effective therapeutic outcome for patients, particularly those with complex conditions such as multiple sclerosis. In this embodiment, the treatment is formulated as an oil-in-water (O/W) nanoemulsion, where the oil phase containing hydrophobic compounds is finely dispersed within a continuous water phase. The hydrophobic properties of cannabinoids (CBD, CBG, and THC) and Vitamin D are effectively leveraged in the oil phase, ensuring their solubility and stability within the formulation.

Concurrently, the hydrophilic active components, such as Hericium erinaceus (Lion's Mane extract), are seamlessly integrated into the aqueous phase, allowing for efficient dispersion and compatibility with the water-soluble portion of the nanoemulsion.

The utilization of nanoparticle technology in this nanoemulsion system offers a multitude of advantages for drug delivery. In several embodiments, the nanoemulsion provides high drug-loading efficiency, allowing for increased concentrations of active ingredients without compromising the stability of the formulation. This technology is also highly compatible with combination therapies, making it possible to co-deliver multiple bioactive compounds in a single formulation, thereby enhancing the overall therapeutic effect.

The nanoemulsion system further enables controlled release of the active ingredients, allowing for sustained delivery over time and reducing the frequency of administration. This controlled release profile extends systemic circulation of the cannabinoids and other active compounds, maintaining therapeutic levels in the bloodstream for longer periods. Additionally, the nanometer-sized droplets enhance the bioavailability and absorption of the hydrophobic components, facilitating efficient transport across biological membranes and improving the onset of therapeutic effects.

Moreover, the enhanced targeted delivery capabilities of nanoparticle technology contribute to the improved efficacy of the treatment, as the nanoemulsion can be engineered to increase the penetration of active ingredients into specific tissues, such as the central nervous system. This targeted delivery is particularly advantageous for conditions like multiple sclerosis, where therapeutic compounds need to cross the blood-brain barrier to exert their effects on inflamed and damaged neural tissues. By offering a stable, high-efficiency delivery platform, the oil-in-water nanoemulsion provides a versatile and effective solution for managing complex neurological disorders, optimizing both the safety and efficacy of the treatment.

The present disclosure describes a process for manufacturing a composition for the treatment of multiple sclerosis. The process is designed to produce a stable and highly bioavailable nanoemulsion formulation suitable for oral administration, specifically in the form of drops. The manufacturing steps are detailed as follows:
a. Combining CBD, CBG, and THC isolates to form an active cannabinoid mixture: The process begins by selecting and accurately measuring the isolated cannabinoids-cannabidiol (CBD), cannabigerol (CBG), and tetrahydrocannabinol (THC). These cannabinoids are combined to create an active mixture that serves as the primary therapeutic component of the formulation. The selected ratios and concentrations of each cannabinoid are tailored to provide a balanced therapeutic profile, addressing the diverse symptoms of multiple sclerosis, including neuroinflammation, spasticity, and chronic pain.
b. Incorporating herbal extracts into the active cannabinoid mixture to support the neural axis: The active cannabinoid mixture is then blended with herbal extracts, such as Hericium erinaceus (Lion's Mane), which are chosen for their neuroprotective and regenerative properties. The addition of these extracts supports the neural axis by promoting nerve growth factor (NGF) production and enhancing cognitive function. The herbal extracts are incorporated into the mixture under controlled conditions to ensure uniform dispersion and maximize their therapeutic potential.
c. Utilizing at least two emulsifying agents to enhance the bioavailability and stability of the composition for oral drops administration: The process includes the incorporation of at least two emulsifying agents, such as Polysorbate 80 and Sorbitan 20. These emulsifiers are critical for stabilizing the oil-in-water emulsion, reducing surface tension, and preventing phase separation. By improving the solubility of the hydrophobic cannabinoids, the emulsifiers enhance the bioavailability of the active ingredients, ensuring consistent absorption when administered as oral drops.
d. Subjecting the mixture to ultrasonic cavitation or high-pressure homogenization to create a nanoemulsion: The combined mixture of cannabinoids, herbal extracts, and emulsifying agents is subjected to ultrasonic cavitation or high-pressure homogenization. These processes generate intense shear forces, effectively reducing the particle size of the active ingredients to the nanometer scale. The resulting nanoemulsion provides a uniform and consistent dispersion of cannabinoids and herbal extracts, significantly increasing the surface area and solubility of the hydrophobic compounds. This nanoscale emulsion improves the stability of the formulation, enhances the bioavailability of the active ingredients, and ensures efficient delivery of therapeutic compounds.
e. Formulating the resulting nanoemulsion into a solution for oral drops administration: The final step involves formulating the nanoemulsion into a solution suitable for oral drops administration. The nanoemulsion is adjusted for pH, viscosity, and flavor to ensure patient acceptability and ease of use. The resulting solution is designed to effectively modulate neurotransmitter levels and reduce neuroinflammation, addressing key symptoms of multiple sclerosis. The oral drop format allows for precise and consistent dosing, enhancing therapeutic efficacy and providing sustained relief from symptoms such as spasticity, pain, and cognitive impairment.

This manufacturing process results in a high-quality, stable, and bioavailable formulation, optimized for the effective management of multiple sclerosis symptoms. The use of advanced nanoemulsion technology ensures that the active ingredients are consistently dispersed, maintaining their therapeutic potency throughout the product's shelf life.

### EXAMPLES

### Pharmaceutical Compositions

The present example discloses a therapeutic agent specifically formulated for the treatment of multiple sclerosis. This agent features a unique composition that leverages a nano platform delivery system incorporating key cannabinoids, including cannabidiol (CBD), tetrahydrocannabinol (THC), and cannabigerol (CBG). The use of nanoemulsion technology enhances the bioavailability of these cannabinoids, ensuring efficient delivery and absorption, while optimizing the therapeutic effects of the composition.

The therapeutic substance is designed for administration to patients diagnosed with multiple sclerosis, utilizing a carefully balanced formulation of active ingredients. The specific cannabinoid concentrations include 100 mg/mL of CBD, known for its potent anti-inflammatory and neuroprotective properties; 50 mg/mL of CBG, which contributes additional neuroprotective effects and immune modulation; and 2.5 mg/mL of THC, incorporated at a low concentration to aid in the relief of spasticity and chronic pain without significant psychoactive effects.

In addition to the cannabinoids, the formulation includes 250 mg/ml of Hericium erinaceus (Lion's Mane extract), a bioactive component recognized for its neuroprotective and regenerative properties, supporting nerve growth and reducing oxidative stress. The composition also incorporates 0.13 mg/ml of Vitamin D, which plays a critical role in immune regulation and supports the health of the myelin sheath, addressing key aspects of multiple sclerosis pathology.

The excipients utilized in the formulation serve to enhance the stability, solubility, and patient acceptability of the product. The chosen excipients include: Medium-chain triglycerides (MCTs): Act as lipidic carriers, solubilizing the hydrophobic cannabinoids and enhancing their absorption.

Alpha Tocopherol (Vitamin E): Functions as an antioxidant, protecting the cannabinoids from oxidative degradation and extending the shelf life of the formulation.

Methylparaben and Propylparaben: Preservatives included to inhibit microbial growth, ensuring the safety and stability of the product during storage. Grape flavor: Added to improve the palatability of the oral drops, enhancing patient adherence. Glycerin: Serves as a humectant, providing a smooth texture and aiding in the solubilization of active ingredients.

Polysorbate 80 and Sorbitan 20: Emulsifying agents that facilitate the creation of a stable oil-in-water nanoemulsion, ensuring uniform dispersion of the cannabinoids and other active components.

Purified water: Used as the aqueous phase in the nanoemulsion, contributing to the consistency and quality of the final product.

This carefully designed composition provides a comprehensive therapeutic approach for managing multiple sclerosis, leveraging the synergistic effects of cannabinoids, herbal extracts, and vitamins. The nano platform technology enhances the delivery and efficacy of the active ingredients, offering targeted relief and supporting the overall health of the central nervous system, making it a promising treatment option for patients with multiple sclerosis.

Formulation 1: Sublingual drops at 100 mg/mL CBD, 50 mg/mL CBG, 2.50 mg/mL THC

| Component | Quantity (mg/mL) | Percentage (%) |
|---|---|---|
| CBD isolate | 100 | 10.00 |
| CBG isolate | 50 | 5.00 |
| THC isolate | 2.50 | 0.25 |
| Vitamin D | 0.13 | 0.013 |
| Hericium erinaceus | 250 | 25.0 |
| Medium-chain Triglycerides | 150 | 15 |
| Alpha Tocopherol (Vit E) | 0.50 | 0.05 |
| Methylparaben | 1.80 | 0.18 |
| Propylparaben | 0.20 | 0.02 |
| Grape flavor | 5.0 | 0.50 |
| Glycerin | 100 | 10 |
| Polysorbate 80 | 80.0 | 8.0 |
| Sorbitan 20 | 30.0 | 3.0 |
| Purified Water | 228.9 | 22.9 |

### Nanoemulsion Process

### Example 1: Sublingual Drops at 100 mg/mL CBD, 2.50 mg/mL THC Isolates and 50 mg/mL CBG

1. Preparation of the Aqueous phase: In a mixing tank, add 22.9 grams of purified water and heat to approximately 90° C. Add 0.18 grams of methylparaben and 0.02 grams of propylparaben to the heated water. Stir until both parabens are fully dissolved in the aqueous phase. Cool between 40° C. and 50° C. Incorporate 10.0 grams of Glycerin, 25.0 grams of Hericium erinaceus, 0.5 grams of grape flavor, and 8.0 grams of Polysorbate 80 into the mixture. Stir until all components are fully integrated into the aqueous phase.

2. Preparation of the Oil phase: In a mixing tank, add 15.0 grams of medium-chain triglycerides (MCT) and heat to approximately 40° C. to 50° C. Incorporate 10.0 grams of CBD isolate, 0.25 grams of THC isolate, 5.00 grams of CBG isolate, 0.05 grams of Alpha Tocopherol (Vitamin E), 3.0 grams of Sorbitan 20, and 0.013 grams of Vitamin D. Stir the mixture until all compounds are fully dissolved.

3. Formation of the preliminary emulsion: Slowly add the heated cannabinoid oils to the aqueous phase while stirring vigorously. Use a high-speed homogenizer to ensure proper emulsification of the cannabinoids within the aqueous phase.

4. Formation of the nanoemulsion: A high-energy ultrasonic homogenizer is used to reduce the droplet size in the preliminary emulsion to 50 to 200 nanometers (nm). The sonication process is carried out in 10-second sonication cycles followed by 10 seconds of rest for a total period of 15 to 20 minutes. During the entire sonication process, the temperature of the emulsion is maintained at 50° C. Then, cool the nanoemulsion at 30° C.

5. Cooling of the nanoemulsion: After the sonication process is completed, the resulting nanoemulsion is cooled until it reaches a temperature below 30° C. while stirring at a low speed between 30 and 50 rpm to ensure uniform droplet distribution during cooling.

6. Dispensing into Primary Packaging: Once the nanoemulsion reaches the target temperature, it is dispensed into a 30 mL dropper graduated bottle amber glass primary container suitable for oral administration.

Formulation 2: Sublingual Drops at 200 mg/mL CBD, 50 mg/mL CBG, 2.5 mg/mL THC.

| Component | Quantity (mg/mL) | Percentage (%) |
|---|---|---|
| CBD isolate | 200 | 20.0 |
| CBG isolate | 50 | 5.0 |
| THC isolate | 2.5 | 0.25 |
| Vitamin D | 0.13 | 0.013 |
| Hericium erinaceus | 250 | 25.0 |
| Medium-chain Triglycerides | 100 | 10.0 |
| Alpha Tocopherol (Vit E) | 0.5 | 0.05 |
| Methylparaben | 1.80 | 0.18 |
| Propylparaben | 0.20 | 0.02 |
| Grape flavor | 5.0 | 0.50 |
| Glycerin | 80 | 8.0 |
| Polysorbate 80 | 70 | 7.0 |
| Sorbitan 20 | 20 | 2.0 |
| Purified Water | 218 | 21.8 |

### Nanoemulsion Process:

### Example 2: Sublingual Drops at 200 mg/mL CBD, 50 mg/mL CBG, and 2.50 mg/mL THC

1. Preparation of the Aqueous phase: In a mixing tank, add 21.8 grams of purified water and heat to approximately 90° C. Add 0.18 grams of Methylparaben and 0.02 grams of Propylparaben to the heated water. Stir until both parabens are fully dissolved in the aqueous phase. Cool between 40° C. to 50° C. Incorporate 8 grams of Glycerin, 25 grams of Hericium erinaceus, 0.5 grams of grape flavor, and 7 grams of Polysorbate 80 into the mixture. Stir until all components are fully integrated into the aqueous phase.

2. Preparation of the Oil phase: In a mixing tank, add 10 grams of medium-chain triglycerides (MCT) and heat to approximately 40° C. to 50° C. Incorporate 20 grams of CBD isolate, 0.25 grams of THC isolate, 5 grams of CBG isolate, 0.05 grams of Alpha Tocopherol (Vitamin E), 2.0 grams of Sorbitan 20, and 0.013 grams of Vitamin D. Stir the mixture until all compounds are fully dissolved.

3. Formation of the preliminary emulsion: Slowly add the heated cannabinoid oils to the aqueous phase while stirring vigorously. Use a high-speed homogenizer to ensure proper emulsification of the cannabinoids within the aqueous phase.

4. Formation of the nanoemulsion: TA high-energy ultrasonic homogenizer is used to reduce the droplet size in the preliminary emulsion to 50 to 200 nanometers (nm). The sonication process is carried out in 10-second sonication cycles followed by 10 seconds of rest for a total period of 15 to 20 minutes. During the entire sonication process, the temperature of the emulsion is maintained at 50° C. Then, cool the nanoemulsion at 30° C.

5. Cooling of the nanoemulsion: After the sonication process is completed, the resulting nanoemulsion is cooled until it reaches a temperature below 30° C. while stirring at a low speed between 30 and 50 rpm to ensure uniform droplet distribution during cooling.

6. Dispensing into Primary Packaging: Once the nanoemulsion reaches the target temperature, it is dispensed into a 30 mL dropper graduated bottle amber glass primary container suitable for oral administration.

1. A composition for the treatment of Multiple sclerosis, comprising:
a) an active principle comprising one cannabinoid;
b) a second active principle comprising one herbal extract;
c) a third active principle comprising a vitamin;
d) two emulsifying agents,
wherein the composition is suitable for oral administration.

2. The composition according to claim 1, wherein the cannabinoids are selected from CBD, THC, and CBG, or combinations thereof.

3. The composition according to claim 2, wherein the CBD, THC, CBG or combinations thereof are present in a concentration in the range of 0.2% to 30.0%.

4. The composition according to claim 1, wherein the herbal extract is in a concentration in the range of 20.0% to 30.0%, and the vitamin is in the range of 0.01% to 0.02%.

5. The composition according to claim 1, wherein the emulsifying agents are Polysorbate 80, Sorbitan 20, or combinations thereof and are present in a concentration in the range of 2.0% to 10.0%.

6. The composition described in claim 1, wherein the composition is a nanoplatform which has an average particle size of less than 200 nm and is formulated to ensure the controlled release of cannabinoids and other active ingredients over a sustained period.

7. The composition of claim 1, wherein the composition is provided as an oral nanoemulsion dosage in the form of drops.

8. The composition according to claim 1, wherein the herbal extract is Hericium erinaceus and the vitamin is Vitamin D.

9. A method for treating multiple sclerosis in a human individual, comprising: administering an effective concentration of the composition of claim 1, wherein the active principle comprises at least two cannabinoids, at least one herbal extract enriched with Vitamins, and at least two emulsifying agents.

10. The method of claim 9, wherein the human subject is selected for treatment based on the diagnosis of Multiple Sclerosis.

11. The method of claim 10, wherein the individual is selected for treatment of multiple sclerosis disorders, including Relapsing-Remitting Multiple Sclerosis (RRMS), Secondary Progressive Multiple Sclerosis (SPMS), Primary Progressive Multiple Sclerosis (PPMS), Progressive-Relapsing Multiple Sclerosis (PRMS).

12. The method of claim 9, further comprises identifying individuals using: medical history, physical examination, laboratory values focused on immunological antibodies profile, symptom diary, neurological and immunological symptoms, and additional neurological test: Expanded Disability Status Scale (EDSS).

13. The method of claim 1, where the individual is identified by having: Fatigue, Vision problems, Motor difficulties, Abnormal sensations, Cognitive problems, Spasticity, Bladder and bowel issues, Speech disturbances, Depression, and Anxiety.

14. The method according to claim 9, wherein the effective concentration is administered as 0.5 mL, every 8 hours for 3 months.

15. The method according to claim 9, wherein the administration of the effective concentration of the composition acts as a monotherapy for treating Multiple Sclerosis.

16. The method of claim 9, wherein the treatment composition alleviates: Fatigue, Vision problems, Motor difficulties, Abnormal sensations, Cognitive problems, Spasticity, Bladder and bowel issues, Speech disturbances, Depression, and anxiety.

17. A process for manufacturing the composition for treating Multiple Sclerosis of claim 1, wherein the process comprises:
a. Combining CBD, CBG, and THC isolate to form an active cannabinoid mixture;
b. Incorporating herbal extracts into the active cannabinoid mixture to support the neural axis;
c. Utilizing at least two emulsifying agents to enhance the bioavailability and stability of the composition for oral drops administration;
d. Subjecting the mixture to ultrasonic cavitation or high-pressure homogenization to create a nanoemulsion, wherein the nanoemulsion provides consistent dispersion of cannabinoids and herbal extract within the composition; and
e. Formulating the resulting nanoemulsion into a solution for oral drops administration, wherein the solution composition is effective in modulating neurotransmitter levels and reducing neuroinflammation associated with Multiple sclerosis symptoms.

18. A therapeutic composition for treating multiple sclerosis of claim 1, comprising:
a. CBD, CBG, and THC in a concentration ranging from 0.25% to 30.0%;
b. Herbal extract in a concentration ranging from 20.0% to 30.0%;
c. Vitamin D in a concentration ranging from 0.01% to 0.02%; and
d. Emulsifying agents in a concentration ranging from 2.0% to 10.0%.

19. A therapeutic composition for treating multiple sclerosis, comprising:
a. CBD at a concentration of 10%;
b. CBG at a concentration of 5%;
c. THC at a concentration of 0.25%;
d. Hericium erinaceus herbal extract at a concentration of 25%;
e. Vitamin D at a concentration of 0.013%;
f. Polysorbate 80 at a concentration of 8%; and
g. Sorbitan 20 at a concentration of 3%,
wherein the composition is formulated for oral drops administration.

20. A therapeutic composition for treating multiple sclerosis, comprising:
a. CBD at a concentration of 20%;
b. CBG at a concentration of 5%;
c. THC at a concentration of 0.25%;
d. Hericium erinaceus herbal extract at a concentration of 25%;
e. Vitamin D at a concentration of 0.013%;
f. Polysorbate 80 at a concentration of 7%; and
g. Sorbitan 20 at a concentration of 2%,
wherein the composition is formulated for oral drops administration.
